(19) United States
(12) Patent Application Publication
(10) Pub. No.: US 2025/0134387 A1
(43) Pub. Date: May 1, 2025

(54) TISSUE CHARACTERIZATION IN ONE OR MORE IMAGES, SUCH AS IN INTRAVASCULAR IMAGES, USING ARTIFICIAL INTELLIGENCE

(71) Applicant: CANON U.S.A., INC., Melville, NY (US)

(72) Inventor: Lampros Athanasiou, Medford, MA (US)

(21) Appl. No.: 18/928,722

(22) Filed: Oct. 28, 2024

Related U.S. Application Data
(60) Provisional application No. 63/594,368, filed on Oct. 30, 2023.

(51) Int. Cl. A61B 5/00; A61B 5/02; G06T 11/00; G06V 10/82; G06V 40/10

(52) U.S. Cl. CPC A61B 5/0084; A61B 5/0066; A61B 5/02007; A61B 5/6852; A61B 5/7267; A61B 5/7425; G06T 11/00; G06V 10/82; G06V 40/10; A61B 2576/02; G06T 2210/41; G06V 2201/03

(57) ABSTRACT

One or more devices, systems, methods and storage mediums for performing intravascular imaging and/or optical coherence tomography (OCT) while detecting and/or characterizing one or more tissues are provided. Examples of applications include imaging, evaluating and diagnosing biological objects, such as, but not limited to, for Gastro-intestinal, cardio and/or ophthalmic applications, and being obtained via one or more optical instruments, such as, but not limited to, optical probes, catheters, capsules and needles (e.g., a biopsy needle). Preferably, the intravascular imaging devices, systems methods and storage mediums include or involve a method, such as, but not limited to, using one image, such as a carpet view, to detect and/or characterize the one or more tissues and/or to perform coregistration. Examples of identified or detected tissues include calcium, lipids, and other types of tissue.

### CROSS-REFERENCE TO RELATED APPLICATION(S)

This application relates, and claims priority, to U.S. Prov. Patent Application Ser. No. 63/594,368, filed Oct. 30, 2023, the disclosure of which is incorporated by reference herein in its entirety.

### FIELD OF THE DISCLOSURE

This present disclosure generally relates to computer imaging, tissue characterization, computer vision, and/or to the field of medical imaging, particularly to devices/apparatuses, systems, methods, and storage mediums for using artificial intelligence (“AI”) for performing tissue characterization in one or more images and/or for using one or more imaging modalities, including but not limited to, angiography, Optical Coherence Tomography (OCT), Multi-modality OCT (MM-OCT), near-infrared fluorescence (NIRF), near-infrared auto-fluorescence (NIRAF), OCT-NIRAF, robot imaging, robot imaging, continuum robot imaging, etc. Examples of OCT applications include imaging, evaluating, and diagnosing biological objects, including, but not limited to, for gastro-intestinal, cardio, and/or ophthalmic applications, and being obtained via one or more optical instruments, including, but not limited to, one or more optical probes, one or more catheters, one or more endoscopes, one or more capsules, and one or more needles (e.g., a biopsy needle). One or more devices, systems, methods and storage mediums for characterizing, examining and/or diagnosing, and/or measuring viscosity of, a sample or object (e.g., tissue, an organ, a portion of a patient, etc.) in artificial intelligence application(s) using an apparatus or system that uses and/or controls one or more imaging modalities are discussed herein.

### BACKGROUND

Fiber optic catheters and endoscopes have been developed to access to internal organs. For example, in cardiology, OCT has been developed to see (e.g., capture and visualize) depth resolved images of vessels with a catheter. The catheter, which may include a sheath, a coil and an optical probe, may be navigated to a coronary artery.

Optical coherence tomography (OCT) is a technique for obtaining high-resolution cross-sectional images of tissues or materials, and OCT enables real time visualization. The aim of the OCT techniques is to measure the time delay of light by using an interference optical system or interferometry, such as via Fourier Transform or Michelson interferometers. A light from a light source delivers and splits into a reference arm and a sample (or measurement) arm with a splitter (e.g., a beamsplitter). A reference beam is reflected from a reference mirror (partially reflecting or other reflecting element) in the reference arm while a sample beam is reflected or scattered from a sample in the sample arm. Both beams combine (or are recombined) at the splitter and generate interference patterns. The output of the interferometer is detected with one or more detectors, such as, but not limited to, photodiodes or multi-array cameras, in one or more devices, such as, but not limited to, a spectrometer (e.g., a Fourier Transform infrared spectrometer). The interference patterns are generated when the path length of the sample arm matches that of the reference arm to within the coherence length of the light source. By evaluating the output beam, a spectrum of an input radiation may be derived as a function of frequency. The frequency of the interference patterns corresponds to the distance between the sample arm and the reference arm. The higher frequencies are, the greater are the differences in path length. Single mode fibers may be used for OCT optical probes, and double clad fibers may be used for fluorescence and/or spectroscopy. A multi-modality system, such as, but not limited to, an OCT, fluorescence, and/or spectroscopy system with an optical probe, is developed to obtain multiple information at the same time.

OCT also allows the evaluation of luminal dimensions and the assessment of vessel wall morphology. As discussed in Li L and Jia T, Optical Coherence Tomography Vulnerable Plaque Segmentation Based on Deep Residual U-Net, Reviews in Cardiovascular medicine, September 2019, the disclosure of which is incorporated by reference herein in its entirety, mainly four different tissue types may be detected in OCT: (1) calcium (appears as a signal poor region with sharply delineated borders), (2) fibrous tissue (appears as a high back-scattering homogeneous area), (3) lipid tissue (appears as a signal poor region with diffused borders or no back-scattering signal), and (4) mixed tissue (appears as a heterogeneous area having characteristics from multiple tissue types).

Traditionally, OCT and plaque characterization is performed manually, a process which is time consuming and has high inter-observer variability due to inexperienced operators.

Additionally, attempts at detecting a lumen and/or a vessel wall and at characterizing tissue using segmentation and decision trees have been performed. However, such a method is time consuming since the method requires every cross sectional OCT frame of the pullback to be processed, and the method does not take into consideration spatial connection of tissue in adjacent frames. Use of a deep learning network to characterize tissue by processing every two-dimensional (2D) frame of a pullback also increases computational time and hardware cost since a dedicated processor or a graphics processing unit (GPU) is needed. Furthermore, characterization of tissue using the above techniques is often dependent on lumen and vessel wall detection, and this limits the accuracy of the tissue characterization when a lumen is not well detected.

Accordingly, it would be desirable to provide at least one imaging or optical apparatus/device, system, method, and storage medium that applies machine learning and/or deep learning to evaluate and characterize a target, sample, or object (e.g., a tissue, an organ, a part of a patient, a vessel, etc.) with a higher success rate when compared to traditional techniques, and to use the one or more results to characterize the target, sample, or object (e.g., a tissue, an organ, a part of a patient, a vessel, etc.) more efficiently. It also would be desirable to provide one or more probe/catheter/robot device techniques and/or structure for characterizing the target, sample, or object (e.g., a tissue, an organ, a part of a patient, a vessel, etc.) for use in at least one optical device, assembly, or system to achieve consistent, reliable detection, and/or characterization results at high efficiency and a reasonable cost of manufacture and maintenance.

### SUMMARY

Accordingly, it is a broad object of the present disclosure to provide imaging (e.g., OCT, NIRF, NIRAF, robots, continuum robots, etc.) apparatuses, systems, methods and storage mediums for using and/or controlling multiple imaging modalities, that apply machine learning, especially deep learning, to evaluate and characterize tissue in one or more images (e.g., intravascular images) with greater or maximum success, and that use the results to achieve tissue characterization more efficiently or with maximum efficiency. It is also a broad object of the present disclosure to provide OCT devices, systems, methods, and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.).

Further, it is a broad object of the present disclosure to provide one or more methods or techniques that operate to one or more of the following: (i) detect one or more tissue types (e.g., calcium, lipids, fibrous tissue, mixed tissue, etc.) automatically in an entire or whole pullback of catheter or probe for one or more intravascular images (such as, but not limited to, OCT images); (ii) reduce computational time to characterize the pullback by processing one image (e.g., one image only may be processed instead of a plurality (e.g., 400) of images, a carpet view image may be constructed and processed, an intravascular image may be constructed and processed, etc.) in one or more embodiments; (iii) provide one or more methods that do not require any segmentation prior to tissue characterization; and/or (iv) perform a more detailed tissue detection or characterization since spatial connection of tissue in adjacent frames is taken into consideration in one or more embodiments. For example, by constructing one image (e.g., a carpet view) from the pullback, the process allows the tissue characteristics of adjacent frames to be viewed more realistically (as one tissue which is measurable in length) and to be better characterized by a machine learning (ML) algorithm(s) and/or deep learning algorithm(s) (e.g., in a case where a hardware and/or processor setting is already present or included) since each pixel characterization is based on the values of its neighborhood or neighboring pixels too in one or more embodiments.

To overcome the aforementioned issues of manually performing OCT and plaque characterization, several methodologies of the present disclosure have been developed which use either the image (pixel based approach(es)) or the signal (A-line based approach(es)) of the OCT. Using the A-line based approaches, a length of a tissue are may be calculated, and/or using the pixel based approaches, a tissue area also may be quantified. One or more A-line based approaches may include features such as those discussed in J. Lee et al., “Fully automated plaque characterization in intravascular OCT images using hybrid convolutional and lumen morphology features,” Sci Rep, vol. 10, no. 1, December 2020, doi: 10.1038/s41598-020-59315-6 and G. van Soest et al., “Atherosclerotic tissue characterization in vivo by optical coherence tomography attenuation imaging,” J Biomed Opt, vol. 15, no. 1, p. 11105, January-February 2010, doi: Artn 011105 Doi 10.1117/1.3280271, the disclosures of which are incorporated by reference herein in their entireties. One or more pixel based approaches may include features such as those discussed in L. S. Athanasiou et al., “Methodology for fully automated segmentation and plaque characterization in intracoronary optical coherence tomography images,” J Biomed Opt, vol. 19, no. 2, p. 026009, February 2014, doi: 10.1117/1.JBO.190.2.026009 and L. S. Athanasiou, M. L. Olender, J. M. de La Torre Hernandez, E. Ben-Assa, and E. R. Edelman, “A deep learning approach to classify atherosclerosis using intracoronary optical coherence tomography,” in Progress in Biomedical Optics and Imaging—Proceedings of SPIE, 2019, vol. 10950. doi: 10.1117/12.2513078, the disclosures of which are incorporated by reference herein in their entireties.

To overcome the aforementioned issues of using segmentation and decision trees, one or more embodiments of the present disclosure may construct an image or images and process the constructed image(s) to detect different tissue types, which minimizes computational time. Additionally, one or more embodiments of the present disclosure may construct the image(s) from the pullback frames (e.g., all of the pullback images), which allows the tissue characterizations of adjacent frames to be viewed more realistically (e.g., as one tissue which is measurable in length) and, therefore, to be better or more efficiently detected by machine learning, deep learning, or other artificial intelligence algorithm(s).

In one or more embodiments, a method for detecting tissue (e.g., calcium, lipids, fibrous tissue, mixed tissue, etc.) in one or more intravascular images (e.g., in one or more OCT images, in one or more carpet view images, in one or more other intravascular images discussed herein, in one or more other intravascular images known to those skilled in the art, etc.) may include one or more of the following features: constructing one image (e.g., a carpet view) while the pullback (e.g., an OCT pullback, an intravascular pullback, etc.) is performed and processing only the one image; detecting all the target tissue (e.g., calcium, lipid, etc.) areas, independent of size, since each tissue (e.g., calcium, lipid, etc.) is analyzed as a connected entity (for example, plaques may be formed and expanded along the artery as an entity and not in individual small segments corresponding to one or more intravascular (e.g., OCT) frames; and constructing and characterizing only one image without the need or use of any pre-required algorithm or performing any other measurements such that the method is free of any error propagation (error which, for example, may be transferred from a pre-tissue detection algorithm to the tissue detection algorithm).

In one or more embodiments of the present disclosure, neural nets may evaluate image(s) faster than any human operator, and neural nets may be deployed across an unlimited number of devices and/or systems. This avoids the issue related to training human operators to evaluate tissue(s) and tissue characteristic(s), and the shorter time required for evaluation reduces the chances of harm to a patient or object, tissue, or specimen by shortening active collection and/or imaging time. Additionally, in one or more embodiments, neural net classifiers may be used to detect specific objects (such as, but not limited to, a catheter sheath, robot components, lipid(s), plaque(s), calcium, calcified areas, etc.) such that more useful information is obtained, evaluated, and used (in comparison with evaluating the tissue(s) or object(s) without a neural net, which provides limited, signal poor, or inaccurate information).

Using artificial intelligence, for example (but not limited to), deep/machine learning, residual learning, a computer vision task (keypoint or object detection and/or image segmentation), using a unique architecture structure of a model or models, using a unique training process, using input data preparation techniques, using input mapping to the model, using post-processing and interpretation of the output data, etc., one or more embodiments of the present disclosure may achieve a better or maximum success rate of tissue or tissue characteristic evaluation(s) without (or with less) user interactions, and may reduce processing and/or prediction time to display tissue or tissue characterization result(s). In the present disclosure, a model may be defined as software that takes images as input and returns predictions for the given images as output. In one or more embodiments, one image may be used as the input to generate accurate, useful output data regarding tissue(s) or tissue characteristic(s). In one or more embodiments a model may be a particular instance of a model architecture (set of parameter values) that has been obtained by model training and selection using a machine (and/or deep) learning and/or optimization algorithm/process. A model may consist or may be comprised of the following parts: an architecture defined by a source code (e.g., a convolutional neural network comprised of layers of parameterized convolution kernels and activation functions, a neural network, a deep neural network(s), a recurrent neural network, etc.) and configuration values (parameters, weights, or features) that are initially set to random values and are then over the course of the training iteratively optimized given data examples (e.g., image-label pairs, tissue data in an image or images, etc.), an objective function (loss function), and an optimization algorithm (optimizer).

Neural networks are a computer system/systems, and take inspiration from how neurons in a brain work. In one or more embodiments, a neural network may consist of or may comprise an input layer, some hidden layers of neurons or nodes, and an output layer. The input layer may be where the values are passed to the rest of the model. In MM-OCT application(s), the input layer may be the place where the transformed OCT data may be passed to a model for evaluation. In one or more embodiments, the hidden layer(s) may be a series of layers that contain or include neurons or nodes that establish connections between the neurons or nodes in the other hidden layers. Through training, the values of each of the connections may be altered so that, due to the training, the system/systems will trigger when the expected pattern is detected. The output layer provides the result(s) of the model. In the case of the MM-OCT application(s), this may be a Boolean (true/false) value for detecting one or more tissues and/or tissue characteristic(s).

In one or more embodiments, a model (which, in one or more embodiments, may be software, software/hardware combination, or a procedure that utilizes one or more machine or deep learning algorithms/procedures/processes that has/have been trained on data to make one or more predictions for future, unseen data) has enough resolution to predict and/or evaluate the tissue(s) and/or characteristic(s) of the tissue(s) with sufficient accuracy depending on the application or procedure being performed. The performance of the model may be further improved by subsequently adding more training data and retraining the model to create a new instance of the model with better or optimized performance. For example, additional training data may include data based on user input, where the user may identify or correct the location of a tissue or tissues and/or a characteristic(s) of the tissue(s) in an image.

In one or more embodiments, an apparatus for detecting and/or characterizing one or more tissues in one or more images may include: one or more processors that operate to: (i) perform a pullback of a catheter or probe and/or obtain one or more images or frames from the pullback of the catheter or probe; (ii) create or construct a Carpet View Image (CVI) based on the one or more images or frames from the pullback or otherwise receive or obtain the CVI; (iii) detect or identify tissue type(s) of one or more tissues shown in the CVI, and/or determine one or more characteristics of the one or more tissues, including whether the one or more tissues is a calcium, a lipid, or another type of tissue; (iv) update the CVI by overlaying information on the CVI to indicate the detected or identified tissue type(s) and/or the determined one or more characteristics of the one or more tissues; and (v) display the updated CVI, or the updated CVI with one or more images or frames from the pullback on a display, or store the updated CVI in a memory. In one or more embodiments, the one or more processors may further operate to one or more of the following: (i) detect one or more tissue types automatically in the pullback of the catheter or the probe for one or more intravascular or Optical Coherence Tomography (OCT) images, where the one or more tissue types include the calcium type, the lipid(s) type, a fibrous tissue type, a mixed tissue type, or the another tissue type; (ii) reduce computational time to characterize the pullback by processing one image only, where the CVI is the one image; (iii) not require any segmentation prior to tissue characterization; (iv) perform a more detailed tissue detection or characterization since a spatial connection or connections of tissue in adjacent frames of the pullback, and/or each pixel characterization is based on values of its neighborhood or neighboring pixels, is taken into consideration and/or since the spatial connection(s) and/or neighboring or neighborhood pixel(s) consideration is better characterized by artificial intelligence, Machine Learning (ML), and/or deep learning networks, structure, or algorithm(s) useable by the one or more processors; (v) use A-line based approaches so that a length of a tissue are is calculated by the one or more processors; and/or (vi) use pixel based approaches so that a tissue area is quantified by the one or more processors. In one or more embodiments, the one or more processors may further operate to perform one or more of the following: display the CVI for further processing and/or display the CVI with one or more intravascular or Optical Coherence Tomography (OCT) images; co-register high texture carpet view areas of the CVI with one or more intravascular or Optical Coherence Tomography (OCT) images; overlay a line on one or more intravascular or Optical Coherence Tomography (OCT) images corresponding to a border indicating the presence of a first type of tissue or the calcium where the line is either a solid line or a dashed or dotted line, and/or overlay a different line on the one or more intravascular or OCT images corresponding to a border indicating the presence of a second type of tissue or the lipid(s) where the different line is the other of the solid line or the dashed or dotted line; and/or display two copies of the CVI, where a first copy of the CVI includes lines overlaid on a first copy of the CVI where each of the lines indicate a border in one or more intravascular or Optical Coherence Tomography (OCT) images and where a second copy of the CVI includes first annotated area(s) corresponding to a first tissue or calcified area(s) and second annotated area(s) corresponding to a second tissue or lipid area(s). In one or more embodiments, the one or more processors may further operate to: in a case where the high texture carpet view areas are detected by the one or more processors, form the high texture carpet view areas due to a presence of sharp edges in the A-line frames or images which represent calcium in the one or more intravascular or OCT images; and in a case where dark homogenous areas are detected by the one or more processors, form or associate corresponding dark homogenous areas to represent lipid(s). The one or more processors may further operate to use AI network(s) and Machine Learning (ML) or other AI-based features to train models to automatically detected calcium and/or lipids based on the use of the high texture carpet view areas representing calcium and based on the dark homogenous areas representing lipid(s). The one or more processors may further operate to use the trained models and/or trained AI network(s) on the CVI only to determine or identify and/or characterize the tissue or tissue characteristics. The one or more processors may further operate to: construct a patch of length L around each pixel of the CVI; extract a set of intensity and texture features from the patch or pixel and classify the pixel to the lipid tissue type, the calcium tissue type, or the another tissue type using artificial intelligence, where the artificial intelligence is one of: Machine Learning (ML), random forests, support vector machines (SVM), and/or another AI-based method, network, or feature; or auto-extract and auto-define a set of intensity and texture features from the patch or pixel using a neural network, convolutional neural network, or other AI-based method or feature and classify the pixel to the lipid tissue type, the calcium tissue type, or the another tissue type; and point, mark, or otherwise indicate, in one or more intravascular or Optical Coherence Tomography (OCT) images, where the calcium and/or lipid starts and ends by using a column part of each corresponding area detected by the one or more processors. The one or more processors may further operate to one or more of the following: (i) set a pixel value of 1; (ii) perform construction of the patch based on the CVI; (iii) perform AI processing by using an AI network or other AI structure to obtain or generate pre-trained classifier(s) or patch feature extraction(s) and to obtain or generate a pre-trained Machine Learning (ML) classifier(s); (iv) identify or characterize whether the pixel being evaluated for the patch construction is a calcium pixel, a lipid pixel, or another type of pixel; (v) perform calcium and lipid pixel translation to a cross sectional intravascular or Optical Coherence Tomography (OCT) image or frame and/or to the one or more intravascular or OCT images; and/or (vi) determine whether the pixel value is less than a value for the number of A-lines x, or by, the number of pullback frames, and, in a case where the pixel value is less than the value for the number of A-lines x, or by, the number of pullback frames, then add 1 to the pixel value and repeat limitations (ii) through (vi) for the next pixel being evaluated, or in a case where the pixel value is greater than or equal to the value for the number of A-lines x, or by, the number of pullback frames, then complete the tissue characterization. The one or more processors may further operate to display results of the tissue characterization completion on the display, store the results in the memory, or use the results to train one or more models or AI-networks to auto-detect or auto-characterize the tissue. The one or more processors may further operate to: indicate a calcium pixel or patch using a solid line and/or indicate a lipid pixel or patch using a dotted or dashed line overlaid on the one or more intravascular or OCT images and/or on the CVI; and/or perform a more detailed tissue detection by taking into consideration a spatial connection or connections of tissue in adjacent frames of the pullback.

In one or more embodiments, the trained model may be one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel and/or including tissue characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s).

In one or more embodiments, an apparatus may have one or more of the following occur: the CVI has dimensions equal to a number of A-lines (A) of each A-line frame times, or by, a number of pullback cross sectional frames or images or by a number of total frames (N), and setting a counter i to a value of 1; and the one or more processors further operate to: (i) determine whether i is less than or equal to the number of pullback frames, N; and (ii) in a case where i is less than or equal to N and until i is more than N, repeat the performance of the following: acquire an A-line frame corresponding to the value for i; perform or apply thresholding or automatic thresholding for the acquired A-line frame or image; summarize each line of the thresholded image or frame, and/or compress the thresholded image or frame in one dimension (1D) by summing columns of the thresholded image or frame so that each line of the thresholded image or frame is in 1D; and add the 1D line to the ith line of the created or constructed CVI, and add 1 to the value for i; and/or (iii) in a case where i is more than N such that all of the pullback frames, N, have been processed, show, reveal, display on a display, and/or store the created or constructed CVI in the memory. The constructed or created CVI may be saved in the memory and/or may be sent to the one or more processors or an artificial intelligence (AI) network for use in AI evaluations or determinations. The one or more processors may further operate to use one or more neural networks or convolutional neural networks to one or more of: load a trained model of CVI images including calcium and or lipid area(s), create or construct the CVI, evaluate whether the counter i is less than or equal to, or greater than, the number of pullback frames N, detect or identify the tissue type(s) in the CVI, apply the thresholding or automatic thresholding, perform the summarizing or compressing of the thresholded image in 1D by summing the columns of the image, perform the addition of the 1D line to the ith line of the CVI, determine whether the detected or identified tissue type(s) is/are accurate or correct, determine the one or more of the characteristics of the tissue(s), identify or detect the one or more tissues, overlay data on the CVI to show the location(s) of the intravascular image(s) and/or to show the areas for the tissue type(s), display the results for the tissue identification/detection or characterization on a display, and/or acquire or receive the image data during the pullback operation of the catheter or the probe. The one or more processors may further operate to use one or more neural networks or convolutional neural networks to one or more of: incorporate image processing and machine learning (ML) or deep learning to automatically identify and locate calcium and lipid(s); create or construct the CVI for the whole pullback and apply ML only to the CVI; create or construct the CVI or another image having dimensions equal to the number of A-lines (A) of each A-line frame, by the number of total frames (N); acquire the first A-line frame and continue to acquire the A-lines of the frame, threshold the image, summarize each A-line of the thresholded image to generate a one-dimensional (1D) signal or line having size A, add or copy the 1D signal or line in or to a first column of the A×N image, and repeat the acquire, threshold, summarize, and add or copy features for all of the pullback frames so that a next 1D signal or line is added or copied in or to the corresponding next column of the A×N image until all subsequent 1D signals or lines are added or copied in or to the corresponding subsequent, respective columns of the A×N image; and reveal, show, or display the CVI or the A×N image, and/or store the created or constructed CVI in the memory, after the last 1D signal or line is copied or added in or to the last column of the A×N image.

In one or more embodiments, an apparatus may include one or more of the following: a light source that operates to produce a light; an interference optical system that operates to: (i) receive and divide the light from the light source into a first light with which an object or sample is to be irradiated and a second reference light, (ii) send the second reference light for reflection off of a reference mirror of the interference optical system, and (iii) generate interference light by causing reflected or scattered light of the first light with which the object or sample has been irradiated and the reflected second reference light to combine or recombine, and to interfere, with each other, the interference light generating one or more interference patterns; and/or one or more detectors that operate to continuously acquire the interference light and/or the one or more interference patterns such that the one or more lumen edges, the one or more stents, and/or the one or more artifacts are detected in the images, and the one or more stents and/or the one or more artifacts are removed from the one or more images.

In one or more embodiments, a method for detecting and/or characterizing one or more tissues in one or more images may include: (i) performing a pullback of a catheter or probe and/or obtaining one or more images or frames from the pullback of the catheter or probe; (ii) creating or constructing a Carpet View Image (CVI) based on the one or more images or frames from the pullback or otherwise receive or obtain the CVI; (iii) detecting or identifying tissue type(s) of one or more tissues shown in the CVI, and/or determining one or more characteristics of the one or more tissues, including whether the one or more tissues is a calcium, a lipid, or another type of tissue; (iv) updating the CVI by overlaying information on the CVI to indicate the detected or identified tissue type(s) and/or the determined one or more characteristics of the one or more tissues; and (v) displaying the updated CVI, or the updated CVI with one or more images or frames from the pullback on a display, and/or storing the updated CVI in a memory. One or more embodiments of a method of the present disclosure may include one or more features of the apparatuses, storage mediums, artificial intelligence structure or algorithms, or other features discussed herein.

In one or more embodiments, a computer-readable storage medium may store at least one program that operates to cause one or more processors to execute a method for detecting and/or characterizing one or more tissues in one or more images, where the method may include: (i) performing a pullback of a catheter or probe and/or obtaining one or more images or frames from the pullback of the catheter or probe; (ii) creating or constructing a Carpet View Image (CVI) based on the one or more images or frames from the pullback or otherwise receive or obtain the CVI; (iii) detecting or identifying tissue type(s) of one or more tissues shown in the CVI, and/or determining one or more characteristics of the one or more tissues, including whether the one or more tissues is a calcium, a lipid, or another type of tissue; (iv) updating the CVI by overlaying information on the CVI to indicate the detected or identified tissue type(s) and/or the determined one or more characteristics of the one or more tissues; and (v) displaying the updated CVI, or the updated CVI with one or more images or frames from the pullback on a display, and/or storing the updated CVI in a memory. One or more embodiments of a storage medium of the present disclosure may include one or more features of the apparatuses, methods, artificial intelligence structure or algorithms, or other features discussed herein.

While one or more embodiments may use one image, it may be advantageous from a safety perspective to have the neural net/network evaluate more than one frame/image to establish the status of a tissue or tissues or a characteristic(s) of the tissue(s) with more certainty/accuracy. In one or more embodiments, the tissue(s) may be evaluated using artificial intelligence structure, such as, but not limited, convolutional neural networks, generative adversarial networks (GANs), neural networks, any other AI structure or feature(s) discussed herein, any other AI network structure(s) known to those skilled in the art, etc. For example, a generator of a generative adversarial network may operate to generate an image(s) that is/are so similar to ground truth image(s) that a discriminator of the generative adversarial network is not able to distinguish between the generated image(s) and the ground truth image(s). The generative adversarial network may include one or more generators and one or more discriminators. Each generator of the generative adversarial network may operate to estimate tissue(s) or tissue characteristic(s) of each image (e.g., a CT image, an OCT image, an IVUS image, a bronchoscopic image, etc.), and each discriminator of the generative adversarial network may operate to determine whether the estimated tissue(s) or tissue characteristic(s) of each image (e.g., a CT image, an OCT image, an IVUS image, a bronchoscopic image, etc.) is estimated (or fake) or ground truth (or real). In one or more embodiments, an AI network, such as, but not limited to, a GAN or a consistent GAN (cGAN), may receive an image or images as an input and may obtain or create a tissue map (e.g., annotated area(s) representing one or more tissue types or characteristics) for each image or images. In one or more embodiments, an AI network may evaluate obtained one or more images (e.g., a CT image, an OCT image, an IVUS image, a bronchoscopic image, etc.), one or more virtual images, and one or more ground truth tissue maps to generate tissue map(s) for the one or more images and/or evaluate the generated tissue map(s). A Three Cycle-Consistent Generative Adversarial Network (3cGAN) may be used to obtain or construct the tissue map(s), evaluate the quality of the tissue map(s).

In one or more embodiments, an artificial intelligence training apparatus using a neural network or other AI-ready network may include: a memory; one or more processors in communication with the memory, the one or more processors operating to: training a classifier or patch feature extraction and training an AI-classifier (e.g., a ML classifier, a DL classifier, etc.). In one or more embodiments of the present disclosure, an apparatus, a system, or a storage medium may use an AI network, a neural network, or other AI-ready network to perform any of the method step(s) or feature(s) discussed herein.

The one or more processors may further operate to use one or more neural networks, convolutional neural networks, recurrent neural networks, a generative adversarial network (GAN), a consistent generative adversarial network (cGAN), a three cycle-consistent generative adversarial network (3cGAN), and/or any other AI architecture discussed herein or known to those skilled in the art to one or more of: load the trained model, select a set of angiography frames or other type of image frames or select one or more intravascular images, identify one or more targets, objects, or samples (or one or more tissues in the targets, objects, or samples), evaluate the tissue(s) and/or tissue characteristic(s), determine whether the tissue(s) or tissue characteristic(s) evaluation is appropriate with respect to given prior knowledge (for example, vessel location and pullback direction, prior tissue location or characteristic information, etc.), modify the evaluation results or the location of the tissue(s) or characteristic(s) of the tissue(s) for each frame, construct an image based on the tissue(s) or characteristic(s) of the tissue(s), perform coregistration of the constructed image with one or more intravascular images (e.g., OCT images, two-dimensional (2D) OCT images, etc.), insert or overlay intravascular image data (e.g., indicators or lines used to represent one or more tissue types, indicators or lines used to represent calcified (e.g., with a solid line) areas, indicators or lines used to represent lipid (e.g., with a dashed or dotted line)) over the constructed image (e.g., a line may represent a border in each of the intravascular images, a line or an outlined area may represent areas corresponding to different tissue(s) (e.g., calcified areas, lipid areas, etc.)), and/or acquire or receive the image data during the pullback operation.

In one or more embodiments, the object, target, or sample may include one or more of the following: a vessel, a target specimen or object, a tissue or tissues, and a patient.

The one or more processors may further operate to perform the coregistration by co-registering an acquired or received angiography image or the constructed image (e.g., a carpet view) and an obtained one or more intravascular images, such as, but not limited to, OCT or IVUS images or frames.

In one or more embodiments, a loaded, trained model may be one or a combination of the following: a segmentation (classification) model, a segmentation model with pre-processing, a segmentation model with post-processing, an object detection (regression) model, an object detection model with pre-processing, an object detection model with post-processing, a combination of a segmentation (classification) model and an object detection (regression) model, a deep convolutional neural network model, a recurrent neural network model with long short-term memory that can take temporal relationships across images or frames into account, a model using feature pyramid(s) that can take different image resolutions into account, a genetic algorithm that operates to breed multiple models for improved performance (as compared with a case where the genetic algorithm is not used), a model using residual learning technique(s), and/or any other model discussed herein or known to those skilled in the art.

In one or more embodiments, the one or more processors may further operate to one or more of the following: (i) display an image for each of one or more imaging modalities on a display, wherein the one or more imaging modalities include one or more of the following: a tomography image; an Optical Coherence Tomography (OCT) image; a fluorescence image; a near-infrared auto-fluorescence (NIRAF) image; a near-infrared auto-fluorescence (NIRAF) image in a predetermined view, a carpet view, and/or an indicator view; a near-infrared fluorescence (NIRF) image, a near-infrared fluorescence (NIRF) image in a predetermined view, a carpet view, and/or an indicator view; a three-dimensional (3D) rendering; a 3D rendering of a vessel; a 3D rendering of a vessel in a half-pipe view or display; a 3D rendering of the object; a lumen profile; a lumen diameter display; a longitudinal view; computer tomography (CT); Magnetic Resonance Imaging (MRI); Intravascular Ultrasound (IVUS); an X-ray image or view; and an angiography view; and (ii) change or update the displays based on the tissue(s) or tissue characteristic(s) evaluation results and/or an updated location of the catheter.

One or more embodiments of a non-transitory computer-readable storage medium storing at least one program for causing a computer to execute a method for training a model using artificial intelligence may be used with any method(s) discussed in the present disclosure, including but not limited to, one or more tissue(s) or tissue characteristic(s) evaluation/determination method(s).

One or more embodiments of any method discussed herein (e.g., training method(s), detecting method(s), imaging or visualization method(s), artificial intelligence method(s), etc.) may be used with any feature or features of the apparatuses, systems, other methods, storage mediums, or other structures discussed herein.

One or more of the artificial intelligence features discussed herein that may be used in one or more embodiments of the present disclosure, includes but is not limited to, using one or more of deep learning, a computer vision task, keypoint detection, a unique architecture of a model or models, a unique training process or algorithm, a unique optimization process or algorithm, input data preparation techniques, input mapping to the model, pre-processing, post-processing, and/or interpretation of the output data as substantially described herein or as shown in any one of the accompanying drawings.

In one or more embodiments, tissue(s) and or characteristic(s) of one or more tissues may be evaluated and determined using an algorithm, such as, but not limited to, the Viterbi algorithm.

One or more embodiments of the present disclosure may track and/or calculate a tissue(s) or tissue characteristic(s) evaluation success rate.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods and one or more storage mediums using OCT and/or other imaging modality technique(s) to perform tissue characterization and to perform coregistration using artificial intelligence, including, but not limited to, deep or machine learning, using results of the tissue detection and/or tissue characterization for performing coregistration, etc., are discussed herein. Further features of the present disclosure will in part be understandable and will in part be apparent from the following description and with reference to the attached drawings.

In accordance with one or more embodiments of the present disclosure, apparatuses and systems, and methods and storage mediums for tissue detection and/or tissue characterization in one or more images may operate to characterize biological objects, such as, but not limited to, blood, mucus, tissue (including different types of tissue), etc.

It should be noted that one or more embodiments of the tissue detection and/or characterization method(s) or feature(s) of the present disclosure may be used in other imaging systems, apparatuses or devices, where images are formed from signal reflection and scattering within tissue sample(s) using a scanning probe. For example, IVUS images may be processed in addition to or instead of OCT images.

One or more embodiments of the present disclosure may be used in clinical application(s), such as, but not limited to, intervascular imaging, atherosclerotic plaque assessment, cardiac stent evaluation, balloon sinuplasty, sinus stenting, arthroscopy, ophthalmology, ear research, veterinary use and research, etc.

In accordance with at least another aspect of the present disclosure, one or more technique(s) discussed herein may be employed to reduce the cost of at least one of manufacture and maintenance of the one or more apparatuses, devices, systems and storage mediums by reducing or minimizing a number of optical components and by virtue of the efficient techniques to cut down cost of use/manufacture of such apparatuses, devices, systems and storage mediums.

According to other aspects of the present disclosure, one or more additional devices, one or more systems, one or more methods and one or more storage mediums using, or for use with, one or more tissue detection and/or tissue characterization techniques are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1A is a schematic diagram showing at least one embodiment of a system that may be used for performing one or multiple imaging modality viewing and control and/or for detecting or identifying tissue(s) and/or tissue characteristic(s) in accordance with one or more aspects of the present disclosure;

FIG. 1B is a schematic diagram illustrating an imaging system for executing one or more steps to process image data and/or for detecting or identifying tissue(s) and/or tissue characteristic(s) in accordance with one or more aspects of the present disclosure;

FIG. 2 is a diagram of at least one embodiment of a catheter that may be used with one or more embodiments for detecting or identifying tissue(s) and/or tissue characteristic(s) in accordance with one or more aspects of the present disclosure;

FIG. 3 is a flowchart of at least one embodiment of a method for constructing a carpet view image (CVI) that may be used in accordance with one or more aspects of the present disclosure;

FIG. 4 shows at least one embodiment of co-registration of carpet view areas or images with one or more intravascular (such as, but not limited to, OCT) images that may be used in accordance with one or more aspects of the present disclosure;

FIG. 5 is a flowchart of at least one embodiment of a method for performing tissue characterization that may be used in accordance with one or more aspects of the present disclosure;

FIG. 6 is at least one embodiment of an application example or image (e.g., marking a lipid tissue as a dotted or dashed line and marking a calcified area as a white line) for at least one method for performing tissue characterization that may be used in accordance with one or more aspects of the present disclosure;

FIG. 7A shows at least one embodiment of an imaging apparatus or system for utilizing one or more imaging modalities and artificial intelligence for identifying tissue, for performing tissue characterization, and/or for performing coregistration in accordance with one or more aspects of the present disclosure;

FIG. 7B shows at least another embodiment of an imaging apparatus or system for utilizing one or more imaging modalities and artificial intelligence for identifying tissue, for performing tissue characterization, and/or for performing coregistration in accordance with one or more aspects of the present disclosure;

FIG. 7C shows at least a further embodiment of an imaging (such as, but not limited to, OCT and NIRF/NIRAF) apparatus or system for utilizing one or more imaging modalities and artificial intelligence for identifying tissue, for performing tissue characterization, and/or for performing coregistration in accordance with one or more aspects of the present disclosure;

FIG. 8 is a flow diagram showing a method of performing an imaging feature, function, or technique in accordance with one or more aspects of the present disclosure;

FIG. 9 shows a schematic diagram of an embodiment of a computer that may be used with one or more embodiments of an apparatus or system or one or more methods discussed herein in accordance with one or more aspects of the present disclosure;

FIG. 10 shows a schematic diagram of another embodiment of a computer that may be used with one or more embodiments of an imaging apparatus or system or methods discussed herein in accordance with one or more aspects of the present disclosure;

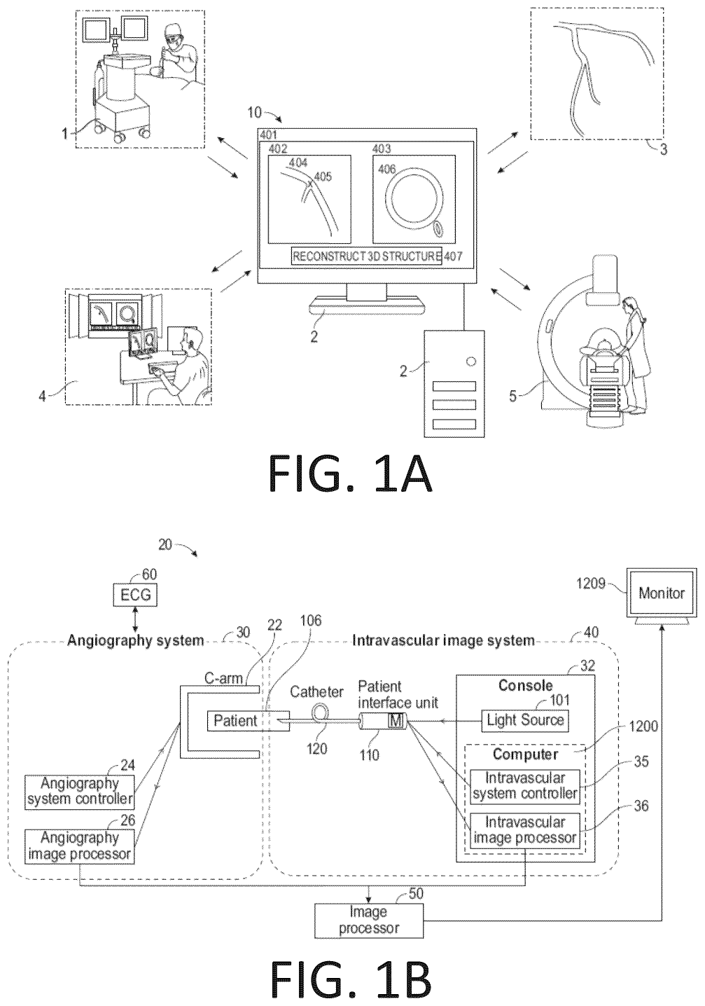

FIG. 1i shows a schematic diagram of at least an embodiment of a system using a computer or processor, a memory, a database, and input and output devices in accordance with one or more aspects of the present disclosure;

FIG. 12 shows a created architecture of or for a regression model(s) that may be used for tissue characterization, tissue detection, and/or any other technique discussed herein in accordance with one or more aspects of the present disclosure;

FIG. 13 shows a convolutional neural network architecture that may be used for tissue characterization, tissue detection, and/or any other technique discussed herein in accordance with one or more aspects of the present disclosure;

FIG. 14 shows a created architecture of or for a regression model(s) that may be used for tissue characterization, tissue detection, and/or any other technique discussed herein in accordance with one or more aspects of the present disclosure; and

FIG. 15 is a schematic diagram of or for a segmentation model(s) that may be used for tissue characterization, tissue detection, and/or any other technique discussed herein in accordance with one or more aspects of the present disclosure.

### DETAILED DESCRIPTION

One or more devices, systems, methods and storage mediums for characterizing tissue, or an object, using one or more imaging techniques or modalities (such as, but not limited to, OCT, fluorescence, IVUS, MRI, CT, NIRF, NIRAF, etc.), and using artificial intelligence for evaluating and detecting tissue types and/or characteristics and/or performing coregistration are disclosed herein. Several embodiments of the present disclosure, which may be carried out by the one or more embodiments of an apparatus, system, method and/or computer-readable storage medium of the present disclosure are described diagrammatically and visually in at least FIGS. 1A through 15 and further discussed below.

One or more embodiments of the present disclosure provide at least one imaging or optical apparatus/device, system, method, and storage medium that applies machine learning and/or deep learning to evaluate and characterize a target, sample, or object (e.g., a tissue, an organ, a part of a patient, a vessel, etc.) with a higher success rate when compared to traditional techniques (for example, techniques performed manually by an operator or user), and to use the one or more results to characterize the target, sample, or object (e.g., a tissue, an organ, a part of a patient, a vessel, etc.) more efficiently. One or more embodiments of the present disclosure may also provide or use one or more probe/catheter/robot device techniques and/or structure for characterizing the target, sample, or object (e.g., a tissue, an organ, a part of a patient, a vessel, etc.) for use in at least one optical device, assembly, or system to achieve consistent, reliable detection, and/or characterization results at high efficiency and a reasonable cost of manufacture and maintenance.

One or more embodiments of the present disclosure provide imaging (e.g., OCT, NIRF, NIRAF, robots, continuum robots, etc.) apparatuses, systems, methods and storage mediums for using and/or controlling multiple imaging modalities, that apply machine learning, especially deep learning, to evaluate and characterize tissue in one or more images (e.g., intravascular images) with greater or maximum success, and that use the results to achieve tissue characterization more efficiently or with maximum efficiency. One or more embodiments of the present disclosure may operate to provide OCT devices, systems, methods, and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.).

Further, one or more embodiments of the present disclosure may provide one or more methods or techniques that operate to one or more of the following: (i) detect one or more tissue types (e.g., calcium, lipids, fibrous tissue, mixed tissue, etc.) automatically in an entire or whole pullback of catheter or probe for one or more intravascular images (such as, but not limited to, OCT images); (ii) reduce computational time to characterize the pullback by processing one image (e.g., one image only may be processed instead of a plurality (e.g., 400) of images, a carpet view image may be constructed and processed, an intravascular image may be constructed and processed, etc.) in one or more embodiments; (iii) provide one or more methods that do not require any segmentation prior to tissue characterization; and/or (iv) perform a more detailed tissue detection or characterization since spatial connection of tissue in adjacent frames is taken into consideration in one or more embodiments. For example, by constructing one image (e.g., a carpet view) from the pullback, the process allows the tissue characteristics of adjacent frames to be viewed more realistically (as one tissue which is measurable in length) and to be better characterized by a machine learning (ML) algorithm(s) and/or deep learning algorithm(s) (e.g., in a case where a hardware and/or processor setting is already present or included) since each pixel characterization is based on the values of its neighborhood or neighboring pixels too in one or more embodiments.

To overcome the aforementioned issues of manually performing OCT and plaque characterization, several methodologies of the present disclosure have been developed which use either the image (pixel based approach(es)) or the signal (A-line based approach(es)) of the OCT. Using the A-line based approaches, a length of a tissue are may be calculated, and/or using the pixel based approaches, a tissue area also may be quantified. One or more A-line based approaches may include features such as those discussed in J. Lee et al., “Fully automated plaque characterization in intravascular OCT images using hybrid convolutional and lumen morphology features,” Sci Rep, vol. 10, no. 1, December 2020, doi: 10.1038/s41598-020-59315-6 and G. van Soest et al., “Atherosclerotic tissue characterization in vivo by optical coherence tomography attenuation imaging,” J Biomed Opt, vol. 15, no. 1, p. 11105, January-February 2010, doi: Artn 011105 Doi 10.1117/1.3280271, the disclosures of which are incorporated by reference herein in their entireties. One or more pixel based approaches may include features such as those discussed in L. S. Athanasiou et al., “Methodology for fully automated segmentation and plaque characterization in intracoronary optical coherence tomography images,” J Biomed Opt, vol. 19, no. 2, p. 026009, February 2014, doi: 10.1117/1.JBO.190.2.026009 and L. S. Athanasiou, M. L. Olender, J. M. de La Torre Hernandez, E. Ben-Assa, and E. R. Edelman, “A deep learning approach to classify atherosclerosis using intracoronary optical coherence tomography,” in Progress in Biomedical Optics and Imaging—Proceedings of SPIE, 2019, vol. 10950. doi: 10.1117/12.2513078, the disclosures of which are incorporated by reference herein in their entireties.

To overcome the aforementioned issues of using segmentation and decision trees, one or more embodiments of the present disclosure may construct an image or images and process the constructed image(s) to detect different tissue types, which minimizes computational time. Additionally, one or more embodiments of the present disclosure may construct the image(s) from the pullback frames (e.g., all of the pullback images), which allows the tissue characterizations of adjacent frames to be viewed more realistically (e.g., as one tissue which is measurable in length) and, therefore, to be better or more efficiently detected by machine learning, deep learning, or other artificial intelligence algorithm(s). While not limited thereto, one or more embodiments of the present disclosure may construct and use only one image or frame to detect one or more tissue types and/or characteristics, which also achieves reduced processing power and time involved such that the embodiments of the present disclosure achieve greater efficiency (compared to methods that may require construction and evaluation of numerous images to evaluate tissue). One or more other embodiments may select or construct an image from less than all of the pullback images for improved efficiency as well in a case where more than one image would be useful for other benefits (e.g., evaluation result(s) confirmation, patient safety, etc.).

In one or more embodiments, a method for detecting tissue (e.g., calcium, lipids, fibrous tissue, mixed tissue, etc.) in one or more intravascular images (e.g., in one or more OCT images, in one or more carpet view images, in one or more other intravascular images discussed herein, in one or more other intravascular images known to those skilled in the art, etc.) may include one or more of the following features: selecting or constructing one image (e.g., a carpet view, one image including two carpet views, only one image, only one carpet view, only one image including two carpet views, selecting an image from a plurality of images, etc.) while the pullback (e.g., an OCT pullback, an intravascular pullback, etc.) is performed and processing only the one image; detecting all the target tissue (e.g., calcium, lipid, etc.) areas, independent of size, since each tissue (e.g., calcium, lipid, etc.) is analyzed as a connected entity (for example, plaques may be formed and expanded along the artery as an entity and not in individual small segments corresponding to one or more intravascular (e.g., OCT) frames; and constructing and characterizing only one image without the need or use of any pre-required algorithm or performing any other measurements such that the method is free of any error propagation (error which, for example, may be transferred from a pre-tissue detection algorithm to the tissue detection algorithm).

In one or more embodiments of the present disclosure, neural nets may evaluate image(s) faster than any human operator, and neural nets may be deployed across an unlimited number of devices and/or systems. This avoids the issue related to training human operators to evaluate tissue(s) and tissue characteristic(s), and the shorter time required for evaluation reduces the chances of harm to a patient or object, tissue, or specimen by shortening active collection and/or imaging time. Additionally, in one or more embodiments, neural net classifiers may be used to detect specific objects (such as, but not limited to, a catheter sheath, robot components, lipid(s), plaque(s), calcium, calcified areas, etc.) such that more useful information is obtained, evaluated, and used (in comparison with evaluating the tissue(s) or object(s) without a neural net, which provides limited, signal poor, or inaccurate information).

Using artificial intelligence, for example (but not limited to), deep/machine learning, residual learning, a computer vision task (keypoint or object detection and/or image segmentation), using a unique architecture structure of a model or models, using a unique training process, using input data preparation techniques, using input mapping to the model, using post-processing and interpretation of the output data, etc., one or more embodiments of the present disclosure may achieve a better or maximum success rate of tissue or tissue characteristic evaluation(s) without (or with less) user interactions, and may reduce processing and/or prediction time to display tissue or tissue characterization result(s). In the present disclosure, a model may be defined as software that takes images as input and returns predictions for the given images as output. In one or more embodiments, one image may be used as the input to generate accurate, useful output data regarding tissue(s) or tissue characteristic(s). In one or more embodiments a model may be a particular instance of a model architecture (set of parameter values) that has been obtained by model training and selection using a machine (and/or deep) learning and/or optimization algorithm/process. A model may consist or may be comprised of the following parts: an architecture defined by a source code (e.g., a convolutional neural network comprised of layers of parameterized convolution kernels and activation functions, a neural network, a deep neural network(s), a recurrent neural network, etc.) and configuration values (parameters, weights, or features) that are initially set to random values and are then over the course of the training iteratively optimized given data examples (e.g., image-label pairs, tissue data in an image or images, etc.), an objective function (loss function), and an optimization algorithm (optimizer).

Neural networks are a computer system/systems, and take inspiration from how neurons in a brain work. In one or more embodiments, a neural network may consist of or may comprise an input layer, some hidden layers of neurons or nodes, and an output layer. The input layer may be where the values are passed to the rest of the model. In MM-OCT application(s), the input layer may be the place where the transformed OCT data may be passed to a model for evaluation. In one or more embodiments, the hidden layer(s) may be a series of layers that contain or include neurons or nodes that establish connections between the neurons or nodes in the other hidden layers. Through training, the values of each of the connections may be altered so that, due to the training, the system/systems will trigger when the expected pattern is detected. The output layer provides the result(s) of the model. In the case of the MM-OCT application(s), this may be a Boolean (true/false) value for detecting one or more tissues and/or tissue characteristic(s).

In one or more embodiments, a model (which, in one or more embodiments, may be software, software/hardware combination, or a procedure that utilizes one or more machine or deep learning algorithms/procedures/processes that has/have been trained on data to make one or more predictions for future, unseen data) has enough resolution to predict and/or evaluate the tissue(s) and/or characteristic(s) of the tissue(s) with sufficient accuracy depending on the application or procedure being performed. The performance of the model may be further improved by subsequently adding more training data and retraining the model to create a new instance of the model with better or optimized performance. For example, additional training data may include data based on user input, where the user may identify or correct the location of a tissue or tissues and/or a characteristic(s) of the tissue(s) in an image.

While one or more embodiments may use one image, it may be advantageous from a safety perspective to have the neural net/network evaluate more than one frame/image to establish the status of a tissue or tissues or a characteristic(s) of the tissue(s) with more certainty/accuracy. In one or more embodiments, the tissue(s) may be evaluated using artificial intelligence structure, such as, but not limited, convolutional neural networks, generative adversarial networks (GANs), neural networks, any other AI structure or feature(s) discussed herein, any other AI network structure(s) known to those skilled in the art, etc. For example, a generator of a generative adversarial network may operate to generate an image(s) that is/are so similar to ground truth image(s) that a discriminator of the generative adversarial network is not able to distinguish between the generated image(s) and the ground truth image(s). The generative adversarial network may include one or more generators and one or more discriminators. Each generator of the generative adversarial network may operate to estimate tissue(s) or tissue characteristic(s) of each image (e.g., a CT image, an OCT image, an IVUS image, a bronchoscopic image, etc.), and each discriminator of the generative adversarial network may operate to determine whether the estimated tissue(s) or tissue characteristic(s) of each image (e.g., a CT image, an OCT image, an IVUS image, a bronchoscopic image, etc.) is estimated (or fake) or ground truth (or real). In one or more embodiments, an AI network, such as, but not limited to, a GAN or a consistent GAN (cGAN), may receive an image or images as an input and may obtain or create a tissue map (e.g., annotated area(s) representing one or more tissue types or characteristics) for each image or images. In one or more embodiments, an AI network may evaluate obtained one or more images (e.g., a CT image, an OCT image, an IVUS image, a bronchoscopic image, etc.), one or more virtual images, and one or more ground truth tissue maps to generate tissue map(s) for the one or more images and/or evaluate the generated tissue map(s). A Three Cycle-Consistent Generative Adversarial Network (3cGAN) may be used to obtain or construct the tissue map(s), evaluate the quality of the tissue map(s).

Turning now to the details of the figures, imaging modalities may be displayed in one or more ways as discussed herein. One or more displays discussed herein may allow a user of the one or more displays to use, control and/or emphasize multiple imaging techniques or modalities, such as, but not limited to, OCT, CT, IVUS, NIRF, NIRAF, etc., and may allow the user to use, control, and/or emphasize the multiple imaging techniques or modalities synchronously.

As shown diagrammatically in FIG. 1A, one or more embodiments for visualizing, emphasizing and/or controlling one or more imaging modalities and artificial intelligence (such as, but not limited to, machine and/or deep learning, residual learning, using results of catheter detection or identification of tissue types and/or characteristics for performing coregistration, etc.) for evaluating and detecting or identifying one or more tissue types and/or tissue characteristics and/or performing coregistration of the present disclosure may be involved with one or more predetermined or desired procedures, such as, but not limited to, medical procedure planning and performance (e.g., Percutaneous Coronary Intervention (PCI)). For example, the system 2 may communicate with the image scanner 5 (e.g., a CT scanner, an X-ray machine, etc.) to request information for use in the medical procedure (e.g., PCI) planning and/or performance, such as, but not limited to, bed positions, and the image scanner 5 may send the requested information along with the images to the system 2 once a clinician uses the image scanner 5 to obtain the information via scans of the patient. In some embodiments, one or more angiograms 3 taken concurrently or from an earlier session are provided for further planning and visualization. The system 2 may further communicate with a workstation such as a Picture Archiving and Communication System (PACS) 4 to send and receive images of a patient to facilitate and aid in the medical procedure planning and/or performance. Once the plan is formed, a clinician may use the system 2 along with a medical procedure/imaging device 1 (e.g., an imaging device, an OCT device, an IVUS device, a PCI device, an ablation device, a 3D structure construction or reconstruction device, etc.) to consult a medical procedure chart or plan to understand the shape and/or size of the targeted biological object to undergo the imaging and/or medical procedure. Each of the medical procedure/imaging device 1, the system 2, the locator device 3, the PACS 4 and the scanning device 5 may communicate in any way known to those skilled in the art, including, but not limited to, directly (via a communication network) or indirectly (via one or more of the other devices such as 1 or 5, or additional flush and/or contrast delivery devices; via one or more of the PACS 4 and the system 2; via clinician interaction; etc.).

In medical procedures, improvement or optimization of physiological assessment is preferable to decide a course of treatment for a particular patient. By way of at least one example, physiological assessment is very useful for deciding treatment for cardiovascular disease patients. In a catheterization lab, for example, physiological assessment may be used as a decision-making tool—e.g., whether a patient should undergo a PCI procedure, whether a PCI procedure is successful, etc. While the concept of using physiological assessment is theoretically sound, physiological assessment still waits for more adaption and improvement for use in the clinical setting(s). This situation may be because physiological assessment may involve adding another device and medication to be prepared, and/or because a measurement result may vary between physicians due to technical difficulties. Such approaches add complexities and lack consistency. Therefore, one or more embodiments of the present disclosure may employ computational fluid dynamics based (CFD-based) physiological assessment that may be performed from imaging data to eliminate or minimize technical difficulties, complexities and inconsistencies during the measurement procedure. To obtain accurate physiological assessment, an accurate 3D structure of the vessel may be reconstructed from the imaging data as disclosed in U.S. Provisional Pat. App. No. 62/901,472, filed on Sep. 17, 2019, the disclosure of which is incorporated by reference herein in its entirety, and as disclosed in U.S. patent application Ser. No. 16/990,800, filed Aug. 11, 2020, the disclosure of which is incorporated by reference herein in its entirety. Additionally or alternatively, the determination or identification of one or more tissue types and/or tissue characteristics operates to provide additional information for physiological assessment.

In at least one embodiment of the present disclosure, a method may be used to provide more accurate 3D structure(s) compared to using only one imaging modality. In one or more embodiments, a combination of multiple imaging modalities may be used, one or more tissue types and/or tissue characteristics may be detected, and coregistration may be processed/performed using artificial intelligence.

One or more embodiments of the present disclosure may apply machine learning, especially deep learning, to detect one or more tissue types and/or tissue characteristics in an image frame without user input(s) that define an area where intravascular imaging pullback occurs. Using artificial intelligence, for example, deep learning, one or more embodiments of the present disclosure may achieve a better or maximum success rate of tissue type(s) and/or tissue characteristic(s) detection from image data without (or with less) user interactions, and may reduce processing and/or prediction time to display coregistration result(s) based on the tissue type(s) and/or tissue characteristic(s) detection result(s) and/or based on the improved image quality obtained when detecting tissue type(s) and/or tissue characteristic(s).

One or more embodiments of the present disclosure may achieve the efficient catheter (or other imaging device) detection of tissue type(s) and/or tissue characteristic(s) and/or efficient coregistration result(s) from image(s). In one or more embodiments, the image data may be acquired during intravascular imaging pullback using a catheter (or other imaging device) that may be visualized in an image. In one or more embodiments, a ground truth identifies a location or locations of the catheter or a portion of the catheter (or of another imaging device or a portion of the another imaging device). In one or more embodiments, a model has enough resolution to predict the tissue type(s) and/or tissue characteristic(s) (e.g., location, size, etc.) in a given image with sufficient accuracy depending on the application or procedure being performed. The performance of the model may be further improved by adding more training data. For example, additional training data may include image annotations, where a user labels or corrects the tissue type(s), tissue characterization(s), and/or catheter detection(s) in each image.

In one or more embodiments, one or more tissue type(s) and/or tissue characteristic(s) may be detected and/or monitored using an algorithm, such as, but not limited to, the Viterbi algorithm.

One or more embodiments may automate characterization of tissue(s) and/or identification of tissue type(s) in images using convolutional neural networks (or other AI structure discussed herein or known to those skilled in the art), and may fully automate frame detection on angiographies, intravascular pullbacks, etc. using training (e.g., offline training) and using applications (e.g., online application(s)) to extract and process frames via deep learning.

One or more embodiments of the present disclosure may track and/or calculate a tissue type(s) and/or tissue characteristic(s) detection or identification success rate.

In at least one further embodiment example, a method of 3D reconstruction without adding any imaging requirements or conditions may be employed. One or more methods of the present disclosure may use intravascular imaging, e.g., IVUS, OCT, etc., and one (1) view of angiography. One or more embodiments may use one image only (e.g., carpet view, a frame of carpet views, another frame or image type discussed herein or known to those skilled in the art, etc.) In the description below, while intravascular imaging of the present disclosure is not limited to OCT, OCT is used as a representative of intravascular imaging for describing one or more features herein.

Referring now to FIG. 1B, shown is a schematic diagram of at least one embodiment of an imaging system 20 for generating an imaging catheter path based on a detected location of an imaging catheter, based on tissue type(s) and/or tissue characteristic(s) detection or identification, and/or a regression line representing the imaging catheter path by using an image frame that is simultaneously acquired during intravascular imaging pullback. The embodiment of FIG. 1B may be used with one or more of the artificial intelligence feature(s) discussed herein. The imaging system 20 may include an angiography system 30, an intravascular imaging system 40, an image processor 50, a display or monitor 1209, and an electrocardiography (ECG) device 60. The angiography system 30 may include an X-ray imaging device such as a C-arm 22 that is connected to an angiography system controller 24 and an angiography image processor 26 for acquiring angiography image frames of an object (e.g., any object that may be imaged using the size and shape of the imaging device, a sample, a vessel, a target specimen or object, etc.) or patient 1o6.

The intravascular imaging system 40 of the imaging system 20 may include a console 32, a catheter 120, and a patient interface unit or PIU 110 that connects between the catheter 120 and the console 32 for acquiring intravascular image frames. The catheter 120 may be inserted into a blood vessel of the patient 1o6 (or inside a specimen or other target object, inside tissue, etc.). The catheter 120 may function as a light irradiator and a data collection probe that is disposed in a lumen of a particular blood vessel, such as, for example, a coronary artery, or in another type of tissue or specimen. The catheter 120 may include a probe tip, one or more markers or radiopaque markers, an optical fiber, and a torque wire. The probe tip may include one or more data collection systems. The catheter 120 may be threaded in an artery of the patient 106 to obtain images of the coronary artery. The patient interface unit 110 may include a motor M inside to enable pullback of imaging optics during the acquisition of intravascular image frames. The imaging pullback procedure may obtain images of the blood vessel. The imaging pullback path may represent the co-registration path, which may be a region of interest or a targeted region of the vessel.

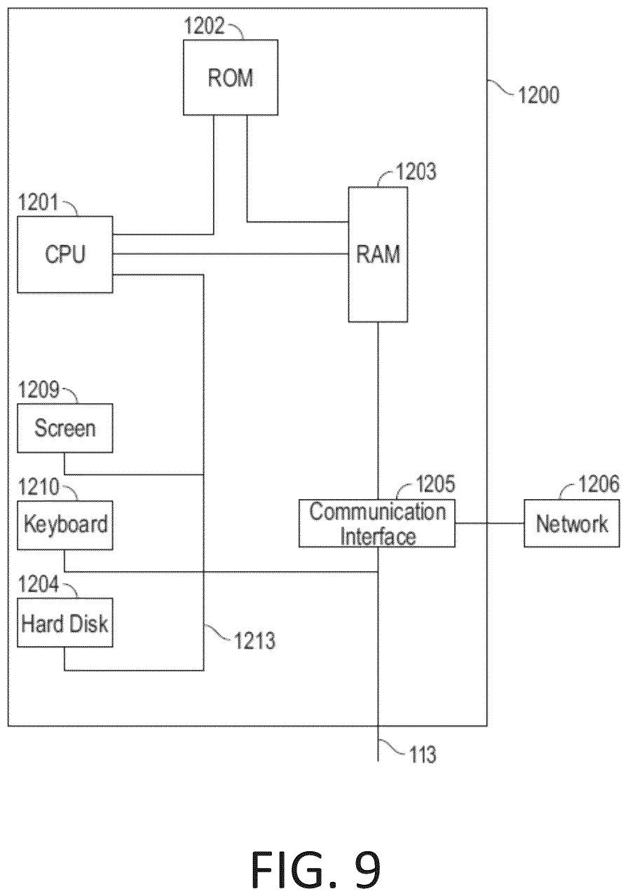

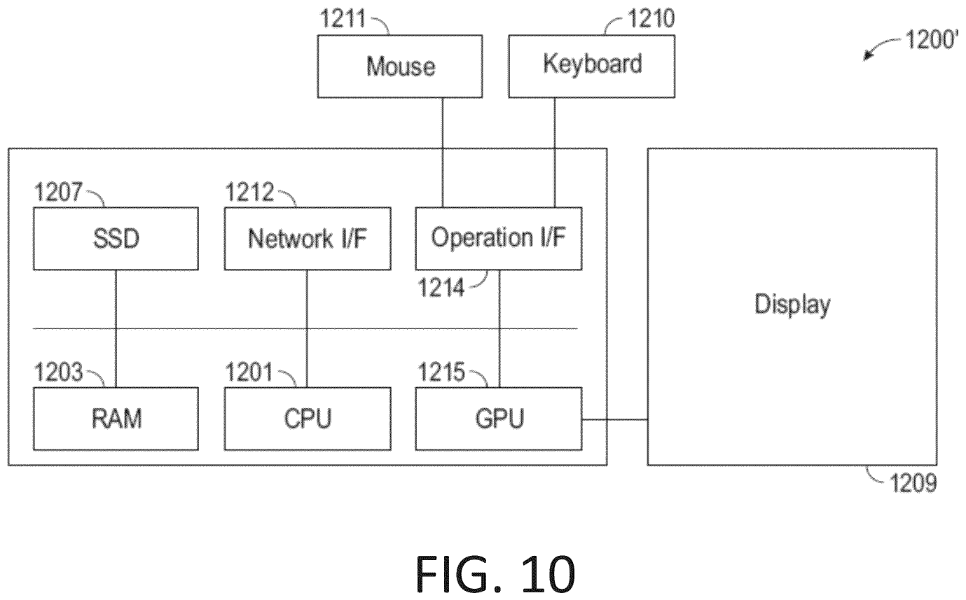

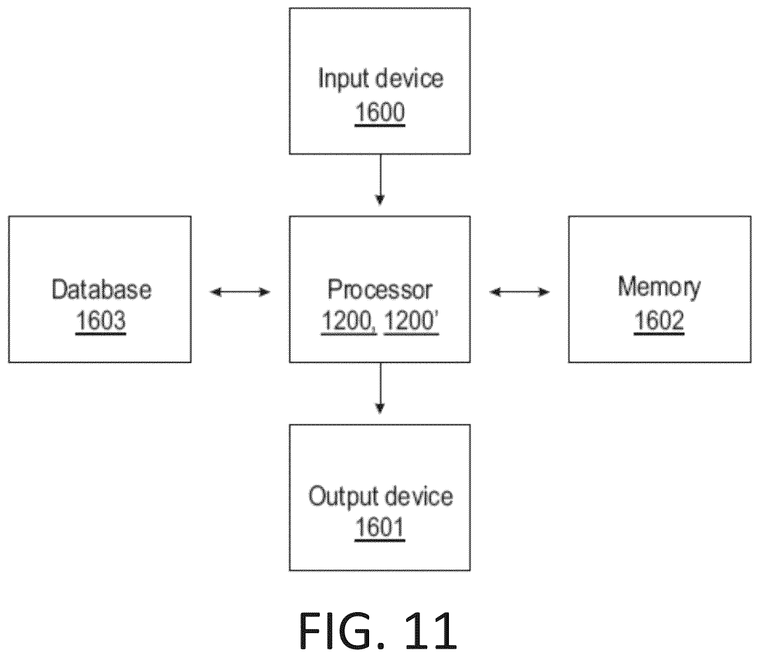

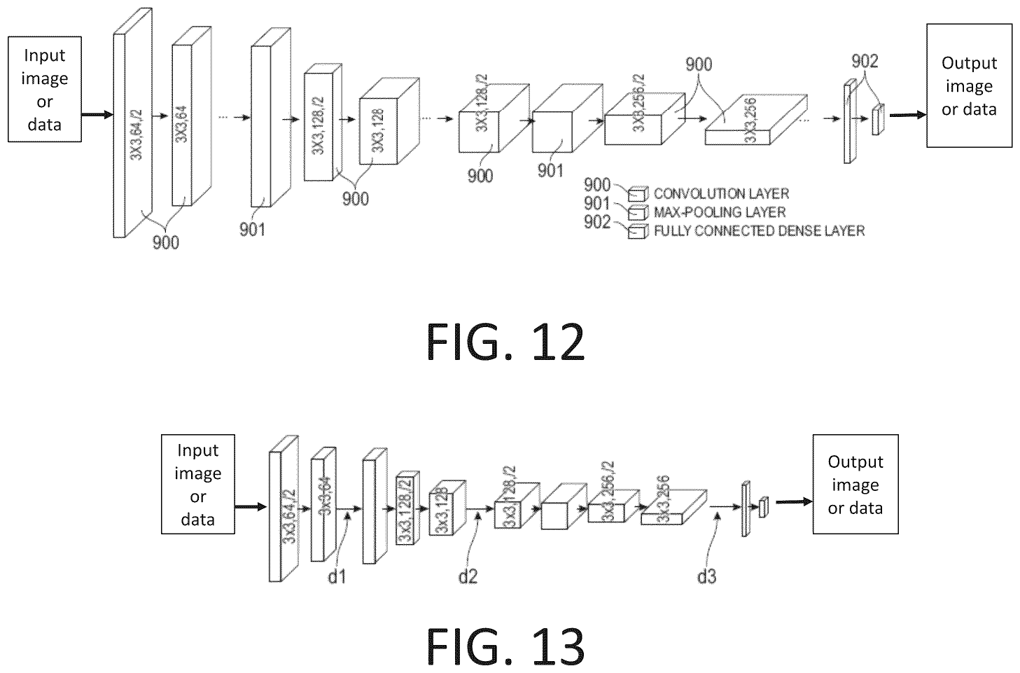

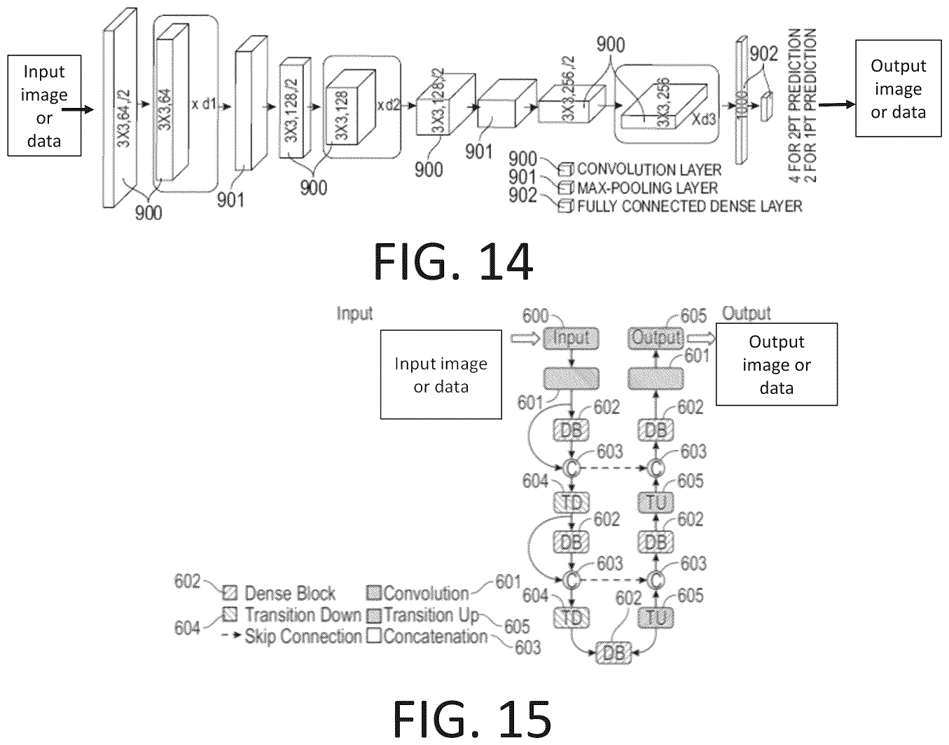

The console 32 may include a light source(s) 101 and a computer 1200. The computer 1200 may include features as discussed herein and below (see e.g., FIG. 9, FIG. 11, etc.), or alternatively may be a computer 1200′ (see e.g., FIG. 10, FIG. 11, etc.) or any other computer or processor discussed herein. In one or more embodiments, the computer 1200 may include an intravascular system controller 35 and an intravascular image processor 36. The intravascular system controller 35 and/or the intravascular image processor 36 may operate to control the motor M in the patient interface unit 11o. The intravascular image processor 36 may also perform various steps for image processing and control the information to be displayed.

Various types of intravascular imaging systems may be used within the imaging system 20. The intravascular imaging system 40 is merely one example of an intravascular imaging system that may be used within the imaging system 20. Various types of intravascular imaging systems may be used, including, but not limited to, an OCT system, a multi-modality OCT system or an IVUS system, by way of example.

The imaging system 20 may also connect to an electrocardiography (ECG) device (or other monitoring device) 60 for recording the electrical activity of the heart (or other organ being monitored, tissue being monitored, specimen being monitored, etc.) over a period of time using electrodes placed on the skin of the patient 106. The imaging system 20 may also include an image processor 50 for receiving angiography data, intravascular imaging data, and data from the ECG device 60 to execute various image-processing steps to transmit to a display 1209 for displaying an angiography image frame with a co-registration path. Although the image processor 50 associated with the imaging system 20 appears external to both the angiography system 20 and the intravascular imaging system 30 in FIG. 1B, the image processor 50 may be included within the angiography system 30, the intravascular imaging system 40, the display 1209, or a stand-alone device. Alternatively, the image processor 50 may not be required if the various image processing steps are executed using one or more of the angiography image processor 26, the intravascular image processor 36 of the imaging system 20, or any other processor discussed herein (e.g., computer 1200, computer 1200′, computer or processor 2, etc.).

To collect data that may be used to train one or more neural nets, one or more features of an OCT device or system (e.g., an MM-OCT device or system, a SS-OCT device or system, etc.) may be used. Collecting a series of OCT images with or without tissue(s) being shown, with one or more tissue types, with one or more tissue characteristics, etc. may result in a plurality (e.g., several thousand) of training images. In one or more embodiments, the data may be labeled based on whether a tissue type was identified or detected, a tissue characteristic was identified or detected, a tissue location was identified or detected, a plurality of tissue types are detected, a plurality of tissue characteristics are detected, etc. (as confirmed by a trained operator or user of the device or system). In one or more embodiments, after at least 30,000 OCT images are captured and labeled, the data may be split into a training population and a test population. In one or more embodiments, data collection may be performed in the same environment or in different environments. For example, during data collection, a flashlight (or any light source) may be used to shine the light down a barrel of an imaging device with no catheter imaging core to confirm that a false positive would not occur in a case where a physician pointed the imaging device at external lights (e.g., operating room lights, a computer screen, etc.). After training is complete, the testing data may be fed through the neural net or neural networks, and the accuracy of the model(s) may be evaluated based on the result(s) of the test data.

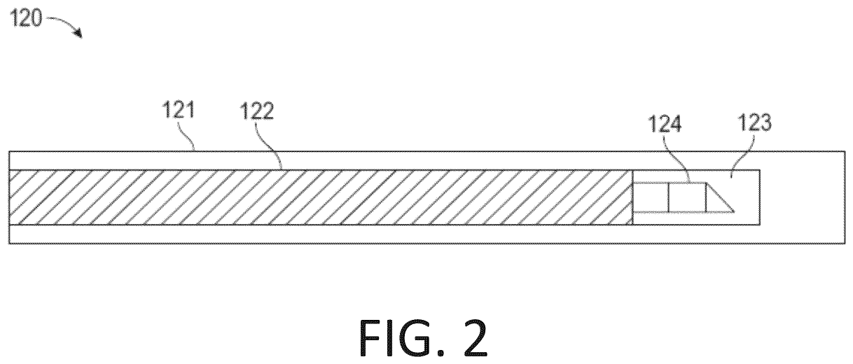

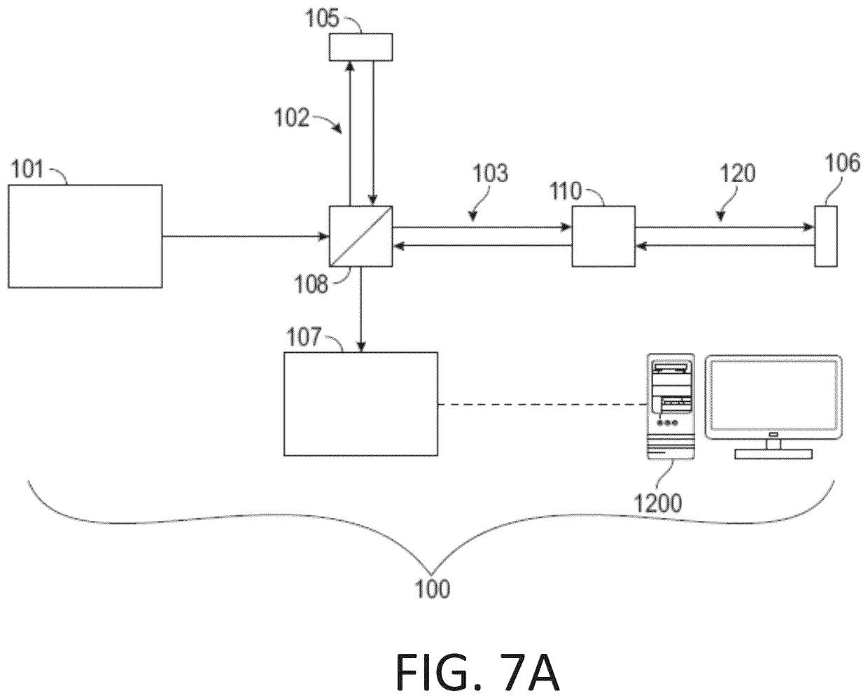

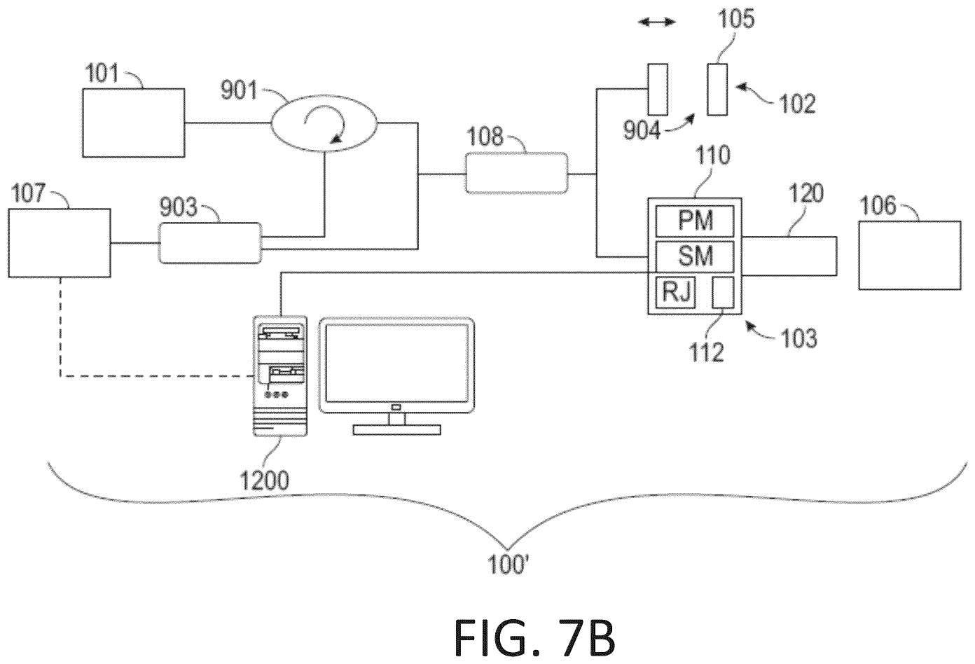

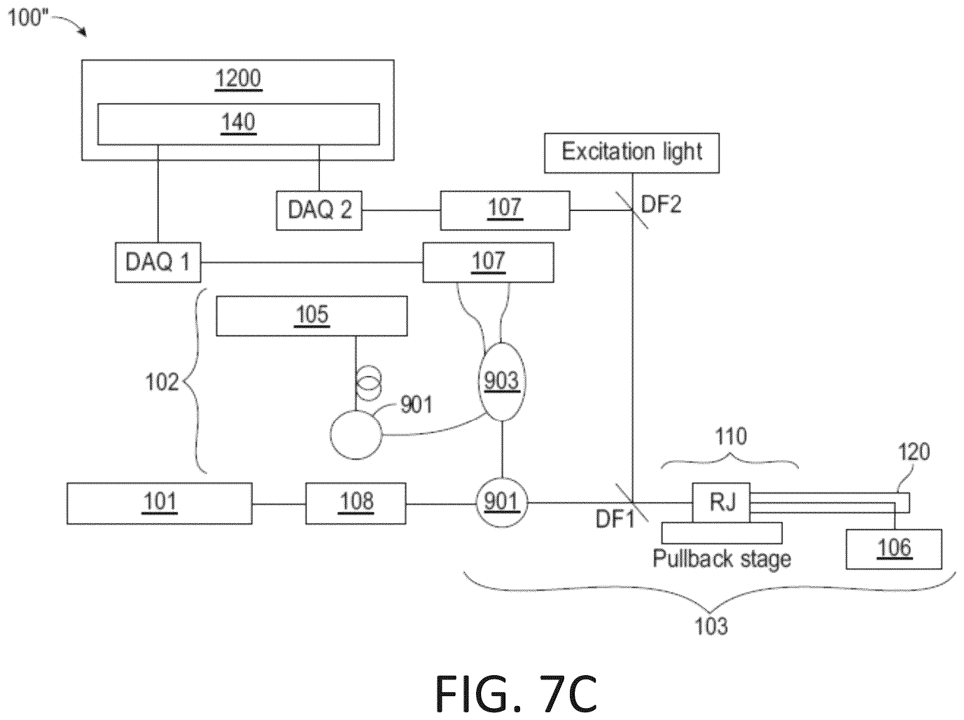

FIG. 2 shows at least one embodiment of a catheter 120 that may be used in one or more embodiments of the present disclosure for obtaining images; for using and/or controlling multiple imaging modalities, that apply machine learning, especially deep learning, to identify one or more tissue type(s) and/or one or more tissue characteristic(s) in an image or frame with greater or maximum success; and for using the results to perform coregistration more efficiently or with maximum efficiency. FIG. 2 shows an embodiment of the catheter 120 including a sheath 121, a coil 122, a protector 123, and an optical probe 124. As shown schematically in FIGS. 7A-7C (discussed further below), the catheter 120 may be connected to a patient interface unit (PIU) 11o to spin the coil 122 with pullback (e.g., at least one embodiment of the PIU 110 operates to spin the coil 122 with pullback). The coil 122 delivers torque from a proximal end to a distal end thereof (e.g., via or by a rotational motor in the PIU 110). In one or more embodiments, the coil 122 is fixed with/to the optical probe 124 so that a distal tip of the optical probe 124 also spins to see an omnidirectional view of the object (e.g., a biological organ, sample or material being evaluated, such as, but not limited to, hollow organs such as vessels, a heart, a coronary artery, etc.). For example, fiber optic catheters and endoscopes may reside in the sample arm (such as the sample arm 103 as shown in one or more of FIGS. 7A-7C discussed below) of an OCT interferometer in order to provide access to internal organs, such as intravascular images, gastro-intestinal tract or any other narrow area, that are difficult to access. As the beam of light through the optical probe 124 inside of the catheter 120 or endoscope is rotated across the surface of interest, cross-sectional images of one or more objects are obtained. In order to acquire imaging data or three-dimensional data, the optical probe 124 is simultaneously translated longitudinally during the rotational spin resulting in a helical scanning pattern. This translation is most commonly performed by pulling the tip of the probe 124 back towards the proximal end and therefore referred to as a pullback.

The catheter 120, which, in one or more embodiments, comprises the sheath 121, the coil 122, the protector 123 and the optical probe 124 as aforementioned (and as shown in FIG. 2), may be connected to the PIU 11o. In one or more embodiments, the optical probe 124 may comprise an optical fiber connector, an optical fiber and a distal lens. The optical fiber connector may be used to engage with the PIU 11o. The optical fiber may operate to deliver light to the distal lens. The distal lens may operate to shape the optical beam and to illuminate light to the object (e.g., the object 106 (e.g., a vessel) discussed herein), and to collect light from the sample (e.g., the object 106 (e.g., a vessel) discussed herein) efficiently. While the target, sample, or object 106 may be a vessel in one or more embodiments, the target, sample, or object 106 may be different from a vessel (and not limited thereto) depending on the particular use(s) or application(s) being employed with the catheter 120.

As aforementioned, in one or more embodiments, the coil 122 delivers torque from a proximal end to a distal end thereof (e.g., via or by a rotational motor in the PIU 110). There may be a mirror at the distal end so that the light beam is deflected outward. In one or more embodiments, the coil 122 is fixed with/to the optical probe 124 so that a distal tip of the optical probe 124 also spins to see an omnidirectional view of an object (e.g., a biological organ, sample or material being evaluated, such as, but not limited to, hollow organs such as vessels, a heart, a coronary artery, etc.). In one or more embodiments, the optical probe 124 may include a fiber connector at a proximal end, a double clad fiber and a lens at distal end. The fiber connector operates to be connected with the PIU 11o. The double clad fiber may operate to transmit & collect OCT light through the core and, in one or more embodiments, to collect Raman and/or fluorescence from an object (e.g., the object 106 (e.g., a vessel) discussed herein, an object and/or a patient (e.g., a vessel in the patient), etc.) through the clad. The lens may be used for focusing and collecting light to and/or from the object (e.g., the object 1o6 (e.g., a vessel) discussed herein). In one or more embodiments, the scattered light through the clad is relatively higher than that through the core because the size of the core is much smaller than the size of the clad.

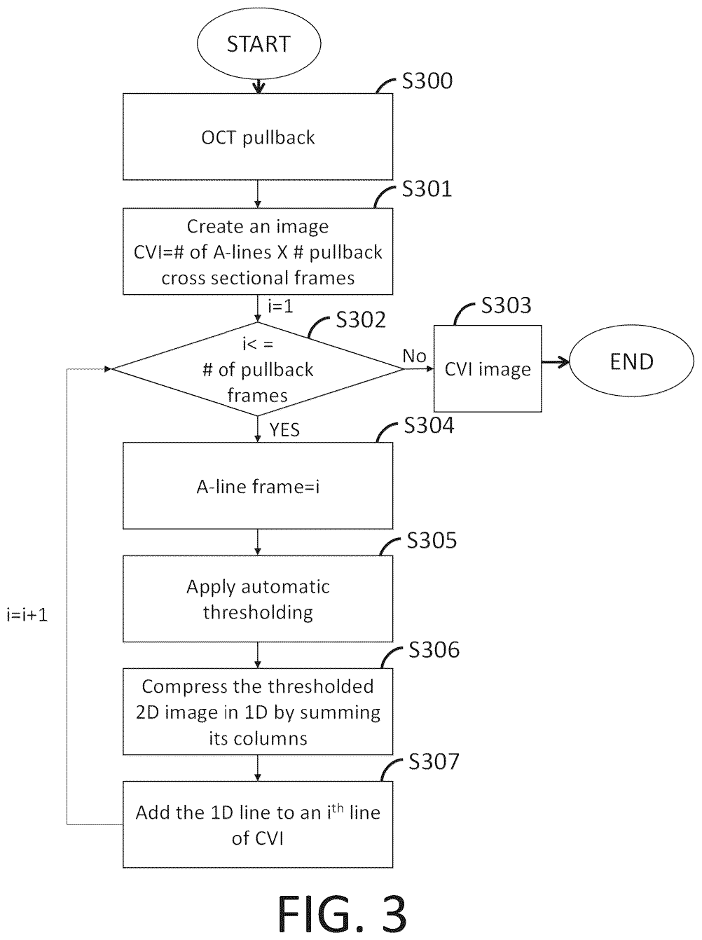

Embodiments of a method or methods for detecting or identifying one or more tissue types and/or tissue characteristics may be used independently or in combination. While not limited to the discussed combination or arrangement, one or more steps may be involved in both of the workflows or processes in one or more embodiments of the present disclosure, for example, as shown in one or more of FIGS. 3-6 and as discussed below.

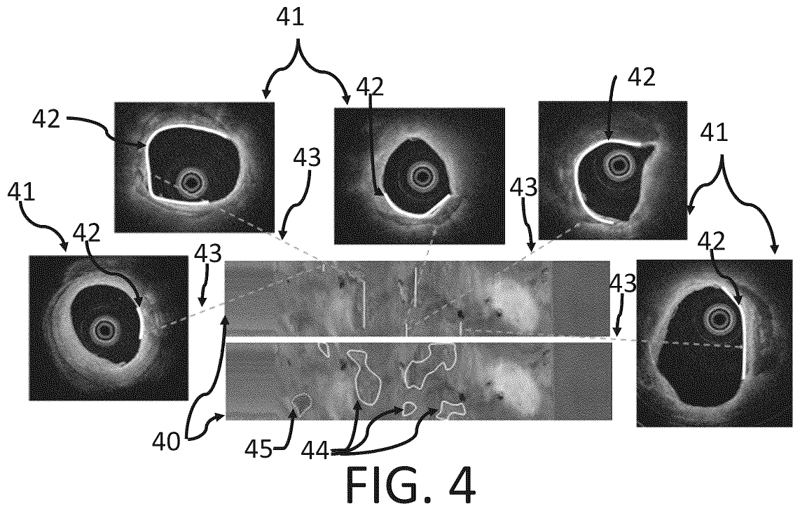

Once trained, a neural network or networks may use a single image (e.g., a single OCT image, an image of a different imaging modality, etc.) to determine or identify one or more tissue types and/or tissue characteristics (or otherwise characterize tissue). FIG. 3 shows at least one embodiment of a method for a construction method for an image that may be used in accordance with one or more aspects of the present disclosure. In one or more embodiments, a single frame or image construction method or methods may include one or more of the following: (i) performing a pullback (e.g., an OCT pullback) and/or obtaining one or more images or frames from the pullback (see e.g., step s300 in FIG. 3); (ii) creating or constructing an image (e.g., a Carpet View Image (CVI), an A×N image, etc.) where the CVI or the image has dimensions equal to a number of A-lines (A) of each A-line frame, times or by a number of pullback cross sectional frames or images or by a number of total frames (N), and setting a counter i to 1 (see e.g., step s301 in FIG. 3); (iii) determining whether i is less than or equal to the number of pullback frames, N; if “YES”, proceed to step S304, and if “NO”, then proceed to step S303 (see e.g., step s302 in FIG. 3); (iv) in step S303 (“NO” in step S302), all of the frames, N, have been processed and the constructed image (e.g., the CVI image) is shown, revealed, displayed, or stored (see e.g., step S303 in FIG. 3), and then the process ends; (v) otherwise, in step S304 (“YES” in step S302), a first A-line frame is acquired (for A-line=i, for the ith iteration, etc.) (see e.g., step S304 in FIG. 3); (vi) thresholding (e.g., automatic thresholding) is performed for the acquired A-line frame (see e.g., step S305 in FIG. 3); (vii) summarizing each line of the thresholded image or frame, and/or compressing the thresholded image (e.g., a thresholded two-dimensional (2D) image) in one dimension (1D) by summing its columns (see e.g., step S306 in FIG. 3); and (viii) adding the 1D line to the ith line of the created or constructed image (e.g., of the created or constructed CVI image) (see e.g., step S307 of FIG. 3), adding 1to i (i=i+1) and then returning to step S302 so that steps S304-S307 may be repeated for each pullback frame or image (until i is greater than N to proceed to step S303 as aforementioned). In one or more embodiments, the data for constructing or creating the image (e.g., the CVI image) and/or being added to the CVI image may be saved in one or more memories or may be sent to one or more processors and/or a neural net (or other AI compatible network or AI-ready network) for use in the AI evaluations/determinations or for use with any other technique(s) or process(es) discussed herein. For example, one or more embodiments of the present disclosure may incorporate image processing and machine learning (ML) to automatically identify and locate calcium and lipids, based on innovative feature(s) of the present disclosure. For example, instead of processing and applying ML in each 2D OCT cross sectional image (400 frames) of a pullback, the method(s) may construct an image, such as a carpet view image (CVI), for the whole pullback and may apply ML only in this image, which operates to radically reduce the computational time and resource requirements (e.g., processor requirements or demands). As aforementioned, one or more embodiments may include constructing an image (A×N) having dimensions equal to the number of A-lines (A) of each A-line frame, by the number of total frames (N). Then, the first A-line frame may be acquired, may be thresholded, and each line of the thresholded image may be summarized resulting to an 1D signal having size A. The 1D signal then may be copied in a first column of the A×N image. The same procedure may be repeated for all of the pullback frames; e.g., the second 1D signal may be copied in the second column of the A×N image, and so on. When the last 1D signal is copied in the last column of the A×N image, the carpet view or views of the pullback may be revealed as aforementioned (see e.g., as shown in FIG. 4).

As shown in FIG. 4, a carpet view or views 40 of a constructed or created image or frame may be displayed and/or used for further processing, and/or may be displayed with one or more intravascular images (e.g., one or more OCT images 41 (see, for example, the five intravascular/OCT images 41 in FIG. 4 around the CVI 40)). FIG. 4 shows co-registration of the high texture carpet view areas with 2D intravascular or OCT images. For the top carpet view image, each white line 43 represents the white inner border line in each of the OCT images 41. For the bottom carpet view image, the annotated areas correspond to calcified (white outline) or lipid (dotted outline) areas. By visually inspecting the carpet view(s) 40, islands of areas having similar texture are viewable (see e.g., bottom CVI view showing the island areas 44 (outlined areas) in FIG. 4). Such high textured areas are formed due to the presence of sharp edges in the A-line images which represent calcium in 2D intravascular or OCT images as show in FIG. 4. Similarly, lipids appear as dark homogeneous areas (see e.g., area 45 outlined using a dashed line as shown in FIG. 4). By using those high texture and strongly homogeneous areas, a ML-based method may be trained to automatically detect calcium and lipids. For each pixel of the CVI image, one or more embodiments may construct a patch of length L around the pixel and may either extract a set of features (e.g., intensity and texture features) and classify the pixel to lipid, calcium, or other tissue type using an ML algorithm (e.g., random forests, support vector machines (SVM), or other type of classification or other AI-based algorithm or other AI network, structure, or feature discussed herein or known to those skilled in the art), or may use a neural network (such as, but not limited to, a CNN) algorithm to auto-extract features (e.g., a network may operate to auto-define extracted feature(s)) and classify the pixel to lipid, calcium, or other tissue type/characteristic. In one or more embodiments, the top and bottom carpet views 40 may be the same carpet view, with the exception being that the top carpet view includes the vertical lines showing the locations of the intravascular (e.g., OCT) images and the bottom carpet view shows or indicates the areas as aforementioned.

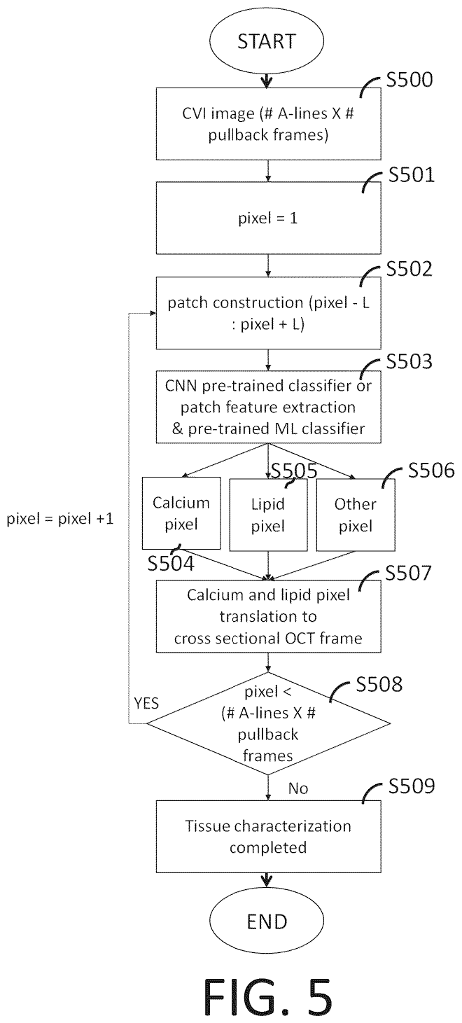

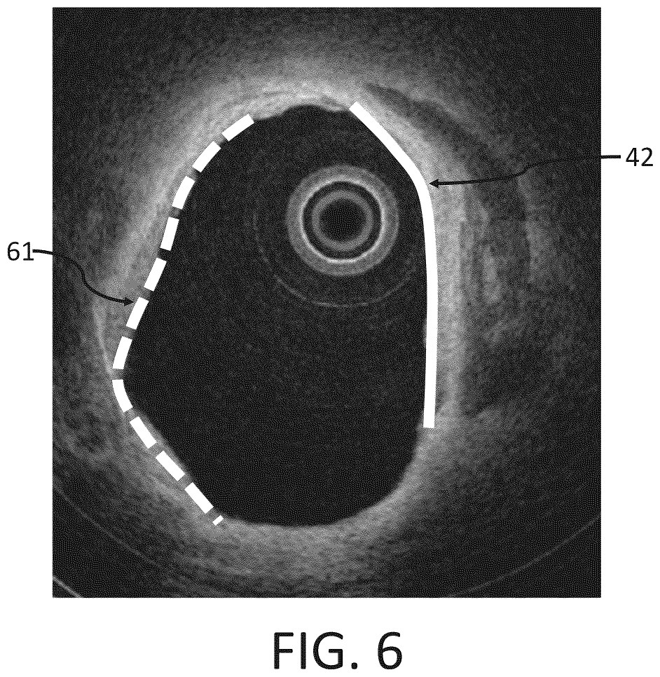

Then, by using the column part of each detected area, one or more embodiments of the present disclosure may point, mark, or otherwise indicate in the cross section OCT frames of the pullback where the calcium and/or lipid starts and ends (see e.g., corresponding white lines 42 in the intravascular/OCT images 41, which are also connected to corresponding lines 43 in the top carpet view 40 via the dashed lines in FIG. 4). Additionally, method(s) may be trained using artificial intelligence to detect more tissue types than calcium and lipid. A flowchart workflow for at least one embodiment of such a ML method is shown in FIG. 5, and at least one application example is shown in FIG. 6.

As aforementioned, once trained, a neural network or networks may use a single image (e.g., a single OCT image, an image of a different imaging modality, etc.) to determine or identify one or more tissue types and/or tissue characteristics (or otherwise characterize tissue). FIG. 5 shows at least one embodiment of a method using AI, ML, or other AI-features for performing tissue characterization that may be used in accordance with one or more aspects of the present disclosure. In one or more embodiments, a tissue characterization method or methods may include one or more of the following: (i) constructing an image (e.g., as outline above for at least the method of FIG. 3) and/or receiving the constructed image (e.g., the CVI image, the image having a number of A-lines times or by a number of pullback frames or images, etc.) (see e.g., step S500 in FIG. 5); (ii) setting a pixel value of 1 (pixel=1) (see e.g., step S501 in FIG. 5); (iii) performing patch construction (for example, pixel−L to pixel+L, where L is the length of the patch around the pixel as aforementioned) on the constructed image (see e.g., step S502 in FIG. 5); (iv) performing AI processing by using a network (e.g., a CNN, a neural network, other type(s) of network(s) discussed herein, other type(s) of network(s) known to those skilled in the art) or other AI-structure to obtain or generate pre-trained classifier(s) or patch feature extraction(s) and to obtain or generate a pre-trained ML classifier(s) (see e.g., step S503 in FIG. 5); (v) identifying or characterizing whether the pixel being evaluated for the patch construction is a calcium pixel (see e.g., step S504 in FIG. 5), a lipid pixel (see e.g., step S505 in FIG. 5), or another type (e.g., another tissue type) of pixel (see e.g., step S506 in FIG. 5); (vi) performing calcium and lipid pixel translation to a cross sectional intravascular (e.g., OCT) image or frame (see e.g., step S507 in FIG. 5); (vii) determining whether the pixel value is less than a value for the number of A-lines x, or by, the number of pullback frames (see e.g., step S508 in FIG. 5), and if “YES”, then adding 1 to the pixel value (pixel=pixel+1) and returning to step S502 to perform steps S502 through S507 for the next pixel being evaluated, or if “NO”, then proceeding to step S509; and (viii) in step S509, completing tissue characterization (see e.g., step S509 in FIG. 5). In one or more embodiments, the results of any of the aforementioned steps, including, but not limited to step S503 and step S509, may be one or more of the following: displayed on a display, stored in a memory for later use (e.g., a trained model may be stored, tissue characterization results may be stored, etc.), further processed (post-processing) (e.g., a trained model may be further improved with additional data, tissue characterization results may be further improved with additional data, etc.), and/or have data that is overlaid on or in the constructed image (e.g., the CVI image, the image having a number of A-lines x, or by, a number of pullback frames, etc.) and/or on or in one or more of the intravascular images (e.g., the OCT images—see e.g., FIGS. 4 and 6).

At least one application embodiment example of using the tissue characterization method of FIG. 5 is shown in FIG. 6. As shown in FIG. 6, a lipid tissue is marked with a dotted or dashed line 61, and the calcium tissue is marked with a whole or solid line 42. Indeed, by using one or more features of the present disclosure, calcium and lipids (and any other type of tissue) may be detected automatically in a whole pullback (e.g., OCT pullback), and the computational time to characterize the pullback may be minimized when using only one image or frame (e.g., the aforementioned carpet view or views) for processing, detecting, and characterizing (instead of 400, for example). Additionally, a more detailed tissue detection may be performed since the spatial connection of tissue in adjacent frames may be taken into consideration.

In one or more embodiments, a model (which, in one or more embodiments, may be software, software/hardware combination, or a procedure that utilizes one or more machine or deep learning algorithms/procedures/processes that has/have been trained on data to make one or more predictions for future, unseen data) has enough resolution to predict and/or evaluate the tissue characterization with sufficient accuracy depending on the application or procedure being performed. The performance of the model may be further improved by subsequently adding more training data and retraining the model to create a new instance of the model with better or optimized performance. For example, additional training data may include data based on user input, where the user may identify or correct the location of a tissue or tissues in an image.

One or more methods, medical imaging devices, Intravascular Ultrasound (IVUS) or Optical Coherence Tomography (OCT) devices, imaging systems, and/or computer-readable storage mediums for evaluating tissue characterization(s) using artificial intelligence may be employed in one or more embodiments of the present disclosure.

In one or more embodiments, an artificial intelligence training apparatus using a neural network or other AI-ready network may include: a memory; one or more processors in communication with the memory, the one or more processors operating to: training a classifier or patch feature extraction and training an AI-classifier (e.g., a ML classifier, a DL classifier, etc.). In one or more embodiments of the present disclosure, an apparatus, a system, or a storage medium may use an AI network, a neural network, or other AI-ready network to perform any of the aforementioned method step(s), including, but not limited to, the steps of FIG. 3 and/or FIG. 5.

The one or more processors may further operate to use one or more neural networks, convolutional neural networks, and/or recurrent neural networks (or other AI-ready or AI compatible network(s)) to one or more of: load the trained model, select a set of image frames, evaluate the tissue characterization, construct the image (e.g., the carpet view image (CVI)), perform the coregistration, overlay data on the image and/or the intravascular image(s) (e.g., the CVI, the OCT image(s), etc.) and acquire or receive the image data during the pullback operation.

In one or more embodiments, the object, target, or sample may include one or more of the following: a vessel, a target specimen or object, one or more tissues, and a patient (or a target or tissue(s) in the patient).

The one or more processors may further operate to perform the coregistration by co-registering an acquired or received angiography image and an obtained one or more intravascular images, such as, but not limited to. Optical Coherence Tomography (OCT) or Intravascular Ultrasound (IVUS) images or frames, and/or by co-registering the carpet view (CVI) with the one or more intravascular images, such as, but not limited to, Optical Coherence Tomography (OCT) or Intravascular Ultrasound (IVUS) images or frames.

In one or more embodiments, a loaded, trained model may be one or a combination of the following: a random forest(s) model, a Support Vector Machine (SVM) model, a segmentation (classification) model, a segmentation model with pre-processing, a segmentation model with post-processing, an object detection (regression) model, an object detection model with pre-processing, an object detection model with post-processing, a combination of a segmentation (classification) model and an object detection (regression) model, a deep convolutional neural network model, a recurrent neural network model with long short-term memory that can take temporal relationships across images or frames into account, a model using feature pyramid(s) that can take different image resolutions into account, a genetic algorithm that operates to breed multiple models for improved performance, and/or a model using residual learning technique(s).

In one or more embodiments, the one or more processors may further operate to one or more of the following: any of the steps of FIG. 3, any of the steps of FIG. 5, any combination of the steps of FIG. 3 and FIG. 5, and/or any combination of the steps or features discussed in the present disclosure.

One or more embodiments of the present disclosure may use other artificial intelligence technique(s) or method(s) for performing training, for splitting data into different groups (e.g., training group, validation group, test group, etc.), or other artificial intelligence technique(s) or method(s), such as, but not limited to, embodiment(s) as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, angiography data and/or intravascular data may be used for training, validation, and/or testing as desired. One or more embodiments of a non-transitory computer-readable storage medium storing at least one program for causing a computer to execute a method for training a model using artificial intelligence may be used with any method(s) discussed in the present disclosure, including but not limited to, one or more tissue type and/or characteristic evaluation/determination method(s).

In one or more embodiments of the present disclosure, an OCT image may be formed in a polar coordinate system from A-lines. Each A-line includes much information about the imaged object, such as, but not limited to: clear indications of artifacts from metal objects (e.g., stents, stent struts, guide wires, etc.) like narrow signal width and/or sharp rising and falling edges; significant difference in signal intensity and shape for unobstructed soft tissue compared to the sheath reflection and other artifacts like wide signal width and a gentle falling edge, tissue type and/or tissue characterization, etc. Each A-line represents a cross-sectional 1D sampling of a target, sample, object, etc., such as, but not limited to, a vessel, along a certain view angle. As an imaging probe or device rotates (e.g., rotates about 0 to about 360 degrees, about 180 degrees to about 360 degrees, about 360 degrees, etc.), the corresponding A-lines form the complete two-dimensional (2D) cross-section of the target, sample, object, etc. (e.g., the vessel) in polar coordinates, which is then converted into Cartesian coordinates to form a tomographical-view (tomo-view) image of the cross-section of the target, sample, object, etc. (e.g., the vessel).

In accordance with at least one aspect of the present disclosure and as aforementioned, one or more additional methods for target or object detection of OCT images are provided herein and are discussed in U.S. patent application Ser. No. 16/414,222, filed on May 16, 2019 and published on Dec. 12, 2019 as U.S. Pat. Pub. No. 2019/0374109, the entire disclosure of which is incorporated by reference herein in its entirety. By way of a few examples, pre-processing may include, but is not limited to, one or more of the following steps: (1) smoothening the 2D image in the Polar coordinate (e.g., using a Gaussian filter, as discussed below, etc.), (2) computing vertical and/or horizontal gradients using a Sobel operator (as otherwise discussed below, etc.), and/or (3) computing binary image using an Otsu method. For example, Otsu's method is an automatic image thresholding technique to separate pixels into two classes, foreground and background, and the method minimizes the intraclass variances between two classes and is equivalent to a globally optimal k-means (see e.g., https://en.wikipedia.org/wiki/Otsu %27s_method). One skilled in the art would appreciate that pre-processing methods other than Otsu's method (such as, but not limited to, Jenks optimization method) may be used in addition to or alternatively to Otsu's method in one or more embodiments.

By way of at least one embodiment example of sheath, a Polar coordinate image (e.g., an OCT Polar coordinate image) may include (from the top side to the bottom side, from the top side to the bottom side of an OCT Polar coordinate image, etc.) a sheath area, and a normal field of view (FOV). In one or more embodiments, the lumen area and edge are within the FOV. Because one or more shapes of the sheath may not be a circle (as may be typically assumed) and because the sheath (and, therefore, the sheath shape) may be attached to or overlap with the lumen or guide wire, it may be useful to separate the sheath from the other shapes (e.g., the lumen, the guide wire, tissue(s), etc.) ahead of time.

By way of at least one embodiment example of computing/finding a peak and a major or maximum gradient edge (e.g., for each A-line), soft tissue and other artifacts may be presented on each A-line by one or more peaks with different characteristics, for example, in one or more embodiments of a lumen OCT image(s) (e.g., a normal lumen OCT image). For example, the soft tissue may have a wide bright region beyond the lumen edge, while the artifacts may produce an abrupt dark shadow area beyond the edge. Due to the high-resolution nature of one or more OCT images, transitions between neighbor A-lines may have signals for both peaks. Such signals may allow one or more method embodiments or processes to obtain more accurate locations of the artifact objects and/or the lumen edges. In one or more embodiments, detection of tissue, lumen edges, artifacts, etc. may be performed as discussed in U.S. Pat. Pub. No. 2021/0174125 A1, published on Jun. 10, 2021, the disclosure of which is incorporated by reference herein in its entirety.

Additionally or alternatively, in one or more embodiments, a principal component analysis method and/or a regional covariance descriptor(s) may be used to detect objects, such as tissue(s). Cross-correlation among neighboring images may be used to improve tissue characterization and/or detection result(s). One or more embodiments may employ segmentation based image processing and/or gradient based edge detection to improve detection result(s).

Additionally or alternatively, automatic thresholding (such as the automatic thresholding of step S305 of FIG. 3) may use an adaptive threshold. For example, for each A-line signal, smoothing may be performed, and a most significant (or maximum) pulse therein may be detected using an adaptive threshold. Based on the mean and the maximum values of the smoothed A-line, a simple threshold may be computed as:

$$Threshold = ( mean + peak ) / 2 ,$$

where “mean” is the average of the smoothed A-line and “peak” is the maximum value of the smoothed A-line.

As a further example, another approach to find the threshold is to find the average between the max peak and min peak as:

$$Threshold = ( min + peak ) / 2.$$

A further alternative approach is to find the threshold based on the max peak as:

$$Threshold = ( peak ) \times 2 / 3.$$

Regardless of the approach, the predetermined or determined threshold is used to detect the most significant pulse corresponding to a tissue or tissues in the specific A-line. Any pulse above the threshold is a tissue pulse candidate. The largest pulse among all the candidates in terms of area under the pulse is considered to be the maximum peak (or the “most significant pulse”). The location of the highest peak of the one dimensional gradient signal along the A-line in the vicinity of the maximum peak is used to identify the exact location of the tissue or tissue(s) point in the smoothed A-line.

Placing together all the tissue points thus detected from all the A-lines forms the tissue edge or line as a function of maximum peak locations vs. A-line indices.

One or more methods or algorithms for performing co-registration and/or imaging may be used in one or more embodiments of the present disclosure, including, but not limited to, the methods or algorithms discussed in U.S. Pat. App. No. 62/798,885, filed on Jan. 30, 2019, discussed in U.S. patent application Ser. No. 17/427,052, filed on Jan. 28, 2020, and discussed in U.S. Pat. Pub. No. 2019/0029624, which application(s) and publication(s) are incorporated by reference herein in their entireties.

Such information and other features discussed herein may be applied to other applications, such as, but not limited to, co-registration, other modalities, etc. Indeed, the useful applications of the features of the present disclosure and of the aforementioned applications and patent publications are not limited to the discussed modalities, images, or medical procedures. Additionally, depending on the involved modalities, images, or medical procedures, one or more control bars may be contoured, curved, or have any other configuration desired or set by a user. For example, in an embodiment using a touch screen as discussed herein, a user may define or create the size and shape of a control bar based on a user moving a pointer, a finger, a stylus, another tool, etc. on the touch screen (or alternatively by moving a mouse or other input tool or device regardless of whether a touch screen is used or not).

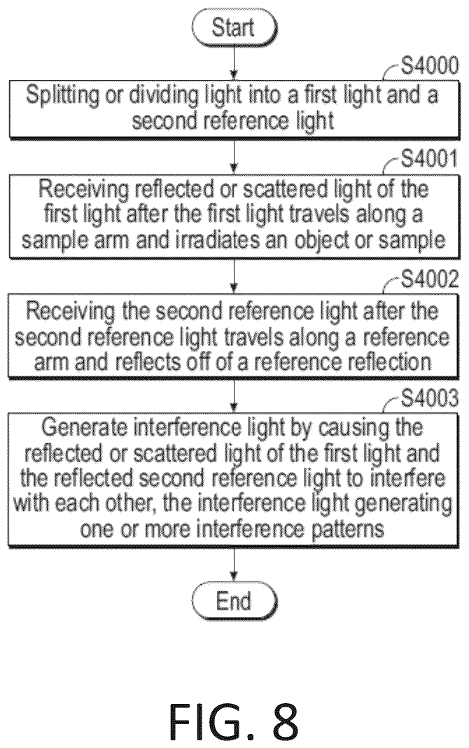

A computer, such as the console or computer 1200, 1200′, may perform any of the steps (e.g., steps S300-S37 of FIG. 3; steps S500-S509 of FIG. 5; steps S4000-S4003 of FIG. 8 discussed further below; etc.) for any system being manufactured or used, including, but not limited to, system 20, system 100, system 100′, system 100″, etc.

In accordance with one or more further aspects of the present disclosure, bench top systems may be utilized for one or more features of the present disclosure, such as, but not limited to, for one or more imaging modalities (such as, but not limited to, angiography, Optical Coherence Tomography (OCT), Multi-modality OCT (MM-OCT), near-infrared auto-fluorescence (NIRAF), near-infrared fluorescence (NIRF), OCT-NIRAF, OCT-NIRF, etc.), and/or for employing one or more additional features discussed herein, including, but not limited to, artificial intelligence processes (e.g., machine or deep learning, residual learning, artificial intelligence (“AI”) co-registration, tissue detection, tissue characterization, etc.) in accordance with one or more aspects of the present disclosure.

FIG. 7A shows an OCT system 100 (as referred to herein as “system 100” or “the system 100”) which may be used for one or more imaging modalities, such as, but not limited to, angiography, Optical Coherence Tomography (OCT), Multi-modality OCT (MM-OCT), near-infrared autofluorescence (NIRAF), near-infrared fluorescence (NIRF), OCT-NIRAF, etc., and/or for employing one or more additional features discussed herein, including, but not limited to, artificial intelligence processes (e.g., machine or deep learning, residual learning, artificial intelligence (“AI”) co-registration, tissue characterization, etc.) in accordance with one or more aspects of the present disclosure. The system 100 comprises a light source 101, a reference arm 102, a sample arm 103, a deflected or deflecting section 108, a reference mirror (also referred to as a “reference reflection”, “reference reflector”, “partially reflecting mirror” and a “partial reflector”) 105, and one or more detectors 107 (which may be connected to a computer 1200). In one or more embodiments, the system 100 may include a patient interface device or unit (“PIU”) 110 and a catheter or probe 120 (see e.g., embodiment examples of a PIU and a catheter as shown in FIGS. 1A-1B, FIG. 2, and/or FIGS. 7A-7C), and the system 100 may interact with an object 106, a patient (e.g., a blood vessel of a patient) 106, a sample, one or more tissues, etc. (e.g., via the catheter 120 and/or the PIU 110). In one or more embodiments, the system 100 includes an interferometer or an interferometer is defined by one or more components of the system 100, such as, but not limited to, at least the light source 101, the reference arm 102, the sample arm 103, the deflecting section 108 and the reference mirror 105.

FIG. 7B shows an example of a system that can utilize the one or more imaging modalities, such as, but not limited to, angiography, Optical Coherence Tomography (OCT), Multi-modality OCT (MM-OCT), near-infrared auto-fluorescence (NIRAF), near-infrared fluorescence (NIRF), OCT-NIRAF, OCT-NIRF, etc., and/or can be used for employing one or more additional features discussed herein, including, but not limited to, artificial intelligence processes (e.g., machine or deep learning, residual learning, artificial intelligence (“AI”) co-registration, tissue detection, tissue characterization, etc.) in accordance with one or more aspects of the present disclosure discussed herein for a bench-top such as for ophthalmic applications. A light from a light source 101 delivers and splits into a reference arm 102 and a sample arm 103 with a deflecting section 108. A reference beam goes through a length adjustment section 904 and is reflected from a reference mirror (such as or similar to the reference mirror or reference reflection 105 shown in FIG. 7A) in the reference arm 102 while a sample beam is reflected or scattered from an object, a patient (e.g., blood vessel of a patient), etc. 106 in the sample arm 103 (e.g., via the PIU 110 and the catheter 120). In one embodiment, both beams combine at the deflecting section 108 and generate interference patterns. In one or more embodiments, the beams go to the combiner 903, and the combiner 903 combines both beams via the circulator 901 and the deflecting section 108, and the combined beams are delivered to one or more detectors (such as the one or more detectors 107). The output of the interferometer is continuously acquired with one or more detectors, such as the one or more detectors 107. The electrical analog signals are converted to the digital signals to analyze them with a computer, such as, but not limited to, the computer 1200 (see FIGS. 7A-7C; also shown in FIGS. 9 and 11 discussed further below), the computer 1200′ (see e.g., FIGS. 10 and 11 discussed further below), the computer 2 (see FIG. 1A), the processors 26, 36, 50 (see FIG. 1B), any other computer or processor discussed herein, etc. Additionally or alternatively, one or more of the computers, CPUs, processors, etc. discussed herein may be used to process, control, update, emphasize, and/or change one or more of imaging modalities, and/or process the related techniques, functions or methods, or may process the electrical signals as discussed above.

The electrical analog signals may be converted to the digital signals to analyze them with a computer, such as, but not limited to, the computer 1200 (see FIGS. 1B and 7A-7C; also shown in FIGS. 9 and 11 discussed further below), the computer 1200′ (see e.g., FIGS. 10-11 discussed further below), the computer 2 (see FIG. 1A), any other processor or computer discussed herein, etc. Additionally or alternatively, one or more of the computers, CPUs, processors, etc. discussed herein may be used to process, control, update, emphasize, and/or change one or more imaging modalities, and/or process the related techniques, functions or methods, or may process the electrical signals as discussed above. In one or more embodiments (see e.g., FIG. 7B), the sample arm 103 includes the PIU 11o and the catheter 120 so that the sample beam is reflected or scattered from the object, patient (e.g., blood vessel of a patient), etc. 106 as discussed herein. In one or more embodiments, the PIU 110 may include one or more motors to control the pullback operation of the catheter 120 (or one or more components thereof) and/or to control the rotation or spin of the catheter 120 (or one or more components thereof) (see e.g., the motor M of FIG. 1B). For example, as best seen in FIG. 7B, the PIU 11o may include a pullback motor (PM) and a spin motor (SM), and/or may include a motion control unit 112 that operates to perform the pullback and/or rotation features using the pullback motor PM and/or the spin motor SM. As discussed herein, the PIU 11o may include a rotary junction (e.g., rotary junction RJ as shown in FIGS. 7B and 7C). The rotary junction RJ may be connected to the spin motor SM so that the catheter 120 may obtain one or more views or images of the object, patient (e.g., blood vessel of a patient, tissue(s), etc.), etc. 106. The computer 1200 (or the computer 1200′, computer 2, any other computer or processor discussed herein, etc.) may be used to control one or more of the pullback motor PM, the spin motor SM and/or the motion control unit 112. An OCT system may include one or more of a computer (e.g., the computer 1200, the computer 1200′, computer 2, any other computer or processor discussed herein, etc.), the PIU 11o, the catheter 120, a monitor (such as the display 1209), etc. One or more embodiments of an OCT system may interact with one or more external systems, such as, but not limited to, an angio system, external displays, one or more hospital networks, external storage media, a power supply, a bedside controller (e.g., which may be connected to the OCT system using Bluetooth technology or other methods known for wireless communication), etc.

In one or more embodiments including the deflecting or deflected section 108 (best seen in FIGS. 7A-7C), the deflected section 108 may operate to deflect the light from the light source 101 to the reference arm 102 and/or the sample arm 103, and then send light received from the reference arm 102 and/or the sample arm 103 towards the at least one detector 107 (e.g., a spectrometer, one or more components of the spectrometer, another type of detector, etc.). In one or more embodiments, the deflected section (e.g., the deflected section 108 of the system 100, 100′, 100″, any other system discussed herein, etc.) may include or may comprise one or more interferometers or optical interference systems that operate as described herein, including, but not limited to, a circulator, a beam splitter, an isolator, a coupler (e.g., fusion fiber coupler), a partially severed mirror with holes therein, a partially severed mirror with a tap, etc. In one or more embodiments, the interferometer or the optical interference system may include one or more components of the system 100 (or any other system discussed herein) such as, but not limited to, one or more of the light source 101, the deflected section 108, the rotary junction RJ, a PIU 11o, a catheter 120, etc. One or more features of the aforementioned configurations of at least FIGS. 1-7B (and/or any other configurations discussed below) may be incorporated into one or more of the systems, including, but not limited to, the system 20, 100, 100′, 100″, etc. discussed herein.

In accordance with one or more further aspects of the present disclosure, one or more other systems may be utilized with one or more of the multiple imaging modalities and related method(s) as disclosed herein. FIG. 7C shows an example of a system 100″ that may utilize the one or more multiple imaging modalities, such as, but not limited to, angiography, Optical Coherence Tomography (OCT), Multi-modality OCT (MM-OCT), near-infrared auto-fluorescence (NIRAF), near-infrared fluorescence (NIRF), OCT-NIRAF, OCT-NIRF, etc., and/or for employing one or more additional features discussed herein, including, but not limited to, artificial intelligence processes (e.g., machine or deep learning, residual learning, artificial intelligence (“AI”) co-registration, tissue detection, tissue characterization, etc.) and/or related technique(s) or method(s) such as for ophthalmic applications in accordance with one or more aspects of the present disclosure. FIG. 7C shows an exemplary schematic of an OCT-fluorescence imaging system 100″, according to one or more embodiments of the present disclosure. An OCT light source 101 (e.g., with a 1.3 μm) is delivered and split into a reference arm 102 and a sample arm 103 with a deflector or deflected section (e.g., a splitter) 1o8, creating a reference beam and sample beam, respectively. The reference beam from the OCT light source 101 is reflected by a reference mirror 105 while a sample beam is reflected or scattered from an object (e.g., an object to be examined, an object, a target, a patient, etc.) 106 through a circulator 901, a rotary junction 90 (“RJ”) and a catheter 120. In one or more embodiments, the fiber between the circulator 901 and the reference mirror or reference reflection 105 may be coiled to adjust the length of the reference arm 102 (best seen in FIG. 7C). Optical fibers in the sample arm 103 may be made of double clad fiber (“DCF”). Excitation light for the fluorescence may be directed to the RJ 90 and the catheter 120, and illuminate the object (e.g., an object to be examined, an object, a patient, etc.) 106. The light from the OCT light source 101 may be delivered through the core of DCF while the fluorescence light emitted from the object (e.g., an object to be examined, an object, a target, a patient, etc.) 106 may be collected through the cladding of the DCF. For pullback imaging, the RJ 90 may be moved with a linear stage to achieve helical scanning of the object (e.g., an object to be examined, an object, a target, a patient, etc.) 106. In one or more embodiments, the RJ 90 may include any one or more features of an RJ as discussed herein. Dichroic filters DF1, DF2 may be used to separate excitation light and the rest of fluorescence and OCT lights. For example (and while not limited to this example), in one or more embodiments, DF1 may be a long pass dichroic filter with a cutoff wavelength of ˜1000 nm, and the OCT light, which may be longer than a cutoff wavelength of DF1, may go through the DF1 while fluorescence excitation and emission, which are a shorter wavelength than the cut off, reflect at DF1. In one or more embodiments, for example (and while not limited to this example), DF2 may be a short pass dichroic filter; the excitation wavelength may be shorter than fluorescence emission light such that the excitation light, which has a wavelength shorter than a cutoff wavelength of DF2, may pass through the DF2, and the fluorescence emission light reflect with DF2. In one embodiment, both beams combine at the deflecting section 108 and generate interference patterns. In one or more embodiments, the beams go to the coupler or combiner 903, and the coupler or combiner 903 combines both beams via the circulator 901 and the deflecting section 1o8, and the combined beams are delivered to one or more detectors (such as the one or more detectors 107; see e.g., the first detector 107 connected to the coupler or combiner 903 in FIG. 7C).

In one or more embodiments, the optical fiber in the catheter 120 operates to rotate inside the catheter 120, and the OCT light and excitation light may be emitted from a side angle of a tip of the catheter 120. After interacting with the object or patient 1o6, the OCT light may be delivered back to an OCT interferometer (e.g., via the circulator 901 of the sample arm 103), which may include the coupler or combiner 903, and combined with the reference beam (e.g., via the coupler or combiner 903) to generate interference patterns. The output of the interferometer is detected with a first detector 107, wherein the first detector 107 may be photodiodes or multi-array cameras, and then may be recorded to a computer (e.g., to the computer 2, the computer 1200 as shown in FIG. 7C, the computer 1200′, or any other computer discussed herein) through a first data-acquisition unit or board (“DAQ1”).

Simultaneously or at a different time, the fluorescence intensity may be recorded through a second detector 107 (e.g., a photomultiplier) through a second data-acquisition unit or board (“DAQ2”). The OCT signal and fluorescence signal may be then processed by the computer (e.g., to the computer 2, the computer 1200 as shown in FIG. 7C, the computer 1200′, or any other computer discussed herein) to generate an OCT-fluorescence data set 140, which includes or is made of multiple frames of helically scanned data. Each set of frames includes or is made of multiple data elements of co-registered OCT and fluorescence data, which correspond to the rotational angle and pullback position.

Detected fluorescence or auto-fluorescence signals may be processed or further processed as discussed in U.S. Pat. App. No. 62/861,888, filed on Jun. 14, 2019, the disclosure of which is incorporated herein by reference in its entirety, as discussed in U.S. patent application Ser. No. 16/898,293, filed Jun. 10, 2020, the disclosure of which is incorporated herein by reference in its entirety, and/or as discussed in U.S. patent application Ser. No. 16/368,510, filed Mar. 28, 2019, the disclosure of which is incorporated herein by reference herein in its entirety.

While not limited to such arrangements, configurations, devices or systems, one or more embodiments of the devices, apparatuses, systems, methods, storage mediums, GUI's, etc. discussed herein may be used with an apparatus or system as aforementioned, such as, but not limited to, for example, the system 20, the system 100, the system 100′, the system 100″, the devices, apparatuses, or systems of FIGS. 1A-1B and 7A-15, any other device, apparatus or system discussed herein, etc. and/or may be used with any AI-ready network discussed herein or known to those skilled in the art. In one or more embodiments, one user may perform the method(s) discussed herein. In one or more embodiments, one or more users may perform the method(s) discussed herein. In one or more embodiments, one or more of the computers, CPUs, processors, etc. discussed herein may be used to process, control, update, emphasize, and/or change one or more of the imaging modalities, and/or process the related techniques, functions or methods, or may process the electrical signals as discussed above.

The light source 101 may include a plurality of light sources or may be a single light source. The light source 101 may be a broadband lightsource, and may include one or more of a laser, an organic light emitting diode (OLED), a light emitting diode (LED), a halogen lamp, an incandescent lamp, supercontinuum light source pumped by a laser, and/or a fluorescent lamp. The light source 101 may be any light source that provides light which may then be dispersed to provide light which is then used for imaging, performing control, viewing, changing, emphasizing methods for imaging modalities, constructing or reconstructing image(s) or structure(s), characterizing tissue, and/or any other method discussed herein. The light source 101 may be fiber coupled or may be free space coupled to the other components of the apparatus and/or system 100, 100′, 100″, the devices, apparatuses or systems of FIGS. 1A-1B and 7A-15, or any other embodiment discussed herein. As aforementioned, the light source 101 may be a swept-source (SS) light source.

Additionally or alternatively, the one or more detectors 107 may be a linear array, a charge-coupled device (CCD), a plurality of photodiodes or some other method of converting the light into an electrical signal. The detector(s) 107 may include an analog to digital converter (ADC). The one or more detectors may be detectors having structure as shown in one or more of FIGS. 1A-1B and 7A-15 and as discussed herein.

In accordance with one or more aspects of the present disclosure, one or more methods for performing imaging are provided herein. FIG. 8 illustrates a flow chart of at least one embodiment of a method for performing imaging. The method(s) may include one or more of the following: (i) splitting or dividing light into a first light and a second reference light (see step S4000 in FIG. 8); (ii) receiving reflected or scattered light of the first light after the first light travels along a sample arm and irradiates an object (see step S4001 in FIG. 8); (iii) receiving the second reference light after the second reference light travels along a reference arm and reflects off of a reference reflection (see step S4002 in FIG. 8); and (iv) generating interference light by causing the reflected or scattered light of the first light and the reflected second reference light to interfere with each other (for example, by combining or recombining and then interfering, by interfering, etc.), the interference light generating one or more interference patterns (see step S4003 in FIG. 8). One or more methods may further include using low frequency monitors to update or control high frequency content to improve image quality. For example, one or more embodiments may use multiple imaging modalities, related methods or techniques for same, etc. to achieve improved image quality. In one or more embodiments, an imaging probe may be connected to one or more systems (e.g., the system 100, the system 100′, the system 100″, the devices, apparatuses or systems of FIGS. 1A-1B and 7A-15, any other system or apparatus discussed herein, etc.) with a connection member or interface module. For example, when the connection member or interface module is a rotary junction for an imaging probe, the rotary junction may be at least one of: a contact rotary junction, a lenseless rotary junction, a lens-based rotary junction, or other rotary junction known to those skilled in the art. The rotary junction may be a one channel rotary junction or a two channel rotary junction. In one or more embodiments, the illumination portion of the imaging probe may be separate from the detection portion of the imaging probe. For example, in one or more applications, a probe may refer to the illumination assembly, which includes an illumination fiber (e.g., single mode fiber, a GRIN lens, a spacer and the grating on the polished surface of the spacer, etc.). In one or more embodiments, a scope may refer to the illumination portion which, for example, may be enclosed and protected by a drive cable, a sheath, and detection fibers (e.g., multimode fibers (MMFs)) around the sheath. Grating coverage is optional on the detection fibers (e.g., MMFs) for one or more applications. The illumination portion may be connected to a rotary joint and may be rotating continuously at video rate. In one or more embodiments, the detection portion may include one or more of: a detection fiber, a detector (e.g., the one or more detectors 107, a spectrometer, etc.), the computer 1200, the computer 1200′, the computer 2, any other computer or processor discussed herein, etc. The detection fibers may surround the illumination fiber, and the detection fibers may or may not be covered by a grating, a spacer, a lens, an end of a probe or catheter, etc.

The one or more detectors 107 may transmit the digital or analog signals to a processor or a computer such as, but not limited to, an image processor, a processor or computer 1200, 1200′ (see e.g., FIG. 1B and FIGS. 7A-7C and 9-11), a computer 2 (see e.g., FIG. 1A), any other processor or computer discussed herein, a combination thereof, etc. The image processor may be a dedicated image processor or a general purpose processor that is configured to process images. In at least one embodiment, the computer 1200, 1200′, 2 or any other processor or computer discussed herein may be used in place of, or in addition to, the image processor. In an alternative embodiment, the image processor may include an ADC and receive analog signals from the one or more detectors 107. The image processor may include one or more of a CPU, DSP, FPGA, ASIC, or some other processing circuitry. The image processor may include memory for storing image, data, and instructions. The image processor may generate one or more images based on the information provided by the one or more detectors 107. A computer or processor discussed herein, such as, but not limited to, a processor of the devices, apparatuses, or systems of FIGS. 1-7C, the computer 1200, the computer 1200′, the computer 2, the image processor, and/or any other processor discussed herein or AI-ready network or neural network discussed herein or known to those skilled in the art, may also include one or more components further discussed herein below (see e.g., FIGS. 9-11).

In at least one embodiment, a console or computer 1200, 1200′, a computer 2, any other computer or processor discussed herein, etc. operates to control motions of the RJ via the motion control unit (MCU) 112 or a motor M, acquires intensity data from the detector(s) in the one or more detectors 107, and displays the scanned image (e.g., on a monitor or screen such as a display, screen or monitor 1209 as shown in the console or computer 1200 of any of FIGS. 1B, 7A-7C, 9, and 11 and/or the console 1200′ of FIGS. 10-11 as further discussed below; the computer 2 of FIG. 1A; any other computer or processor discussed herein; etc.). In one or more embodiments, the MCU 112 or the motor M operates to change a speed of a motor of the RJ and/or of the RJ. The motor may be a stepping or a DC servo motor to control the speed and increase position accuracy (e.g., compared to when not using a motor, compared to when not using an automated or controlled speed and/or position change device, compared to a manual control, etc.).

The output of the one or more components of any of the systems discussed herein may be acquired with the at least one detector 107, e.g., such as, but not limited to, photodiodes, Photomultiplier tube(s) (PMTs), line scan camera(s), or multi-array camera(s). Electrical analog signals obtained from the output of the system 100, 100′, 100″, and/or the detector(s) 107 thereof, and/or from the devices, apparatuses, or systems of FIGS. 1A-7C and/or 9-15, are converted to digital signals to be analyzed with a computer, such as, but not limited to, the computer 1200, 1200′. In one or more embodiments, the light source 101 may be a radiation source or a broadband light source that radiates in a broad band of wavelengths. In one or more embodiments, a Fourier analyzer including software and electronics may be used to convert the electrical analog signals into an optical spectrum.

Unless otherwise discussed herein, like numerals indicate like elements. For example, while variations or differences exist between the systems, such as, but not limited to, the system 20, the system 100, the system 100′, the system 100″, or any other device, apparatus or system discussed herein, one or more features thereof may be the same or similar to each other, such as, but not limited to, the light source 101 or other component(s) thereof (e.g., the console 1200, the console 1200′, etc.). Those skilled in the art will appreciate that the light source 101, the motor or MCU 112, the RJ, the at least one detector 107, and/or one or more other elements of the system 100 may operate in the same or similar fashion to those like-numbered elements of one or more other systems, such as, but not limited to, the devices, apparatuses or systems of FIGS. 1A-7C and/or 9-15, the system 100′, the system 100″, or any other system discussed herein. Those skilled in the art will appreciate that alternative embodiments of the devices, apparatuses or systems of FIGS. 1A-7C and/or 9-15, the system 100′, the system 100″, any other device, apparatus or system discussed herein, etc., and/or one or more like-numbered elements of one of such systems, while having other variations as discussed herein, may operate in the same or similar fashion to the like-numbered elements of any of the other systems (or components thereof) discussed herein. Indeed, while certain differences exist between the system 100 of FIG. 7A and one or more embodiments shown in any of FIGS. 1A-6, 7B-7C, and 9-15, for example, as discussed herein, there are similarities. Likewise, while the console or computer 1200 may be used in one or more systems (e.g., the system 100, the system 100′, the system 100″, the devices, apparatuses or systems of any of FIGS. 1A-15, or any other system discussed herein, etc.), one or more other consoles or computers, such as the console or computer 1200′, any other computer or processor discussed herein, etc., may be used additionally or alternatively.

One or more embodiments of the present disclosure may include taking multiple views (e.g., OCT image, ring view, tomo view, anatomical view, etc.), and one or more embodiments may highlight or emphasize NIRF and/or NIRAF. In one or more embodiments, two handles may operate as endpoints that may bound the color extremes of the NIRF and/or NIRAF data in or more embodiments. In addition to the standard tomographic view, the user may select to display multiple longitudinal views. When connected to an angiography system, the Graphical User Interface (GUI) may also display angiography images.

In accordance with one or more aspects of the present disclosure, the aforementioned features are not limited to being displayed or controlled using any particular GUI. In general, the aforementioned imaging modalities may be used in various ways, including with or without one or more features of aforementioned embodiments of a GUI or GUIs. For example, a GUI may show an OCT image with a tool or marker to change the image view as aforementioned even if not presented with a GUI (or with one or more other components of a GUI; in one or more embodiments, the display may be simplified for a user to display set or desired information).

The procedure to select the region of interest and the position of a marker, an angle, a plane, etc., for example, using a touch screen, a GUI (or one or more components of a GUI; in one or more embodiments, the display may be simplified for a user to display the set or desired information), a processor (e.g., processor or computer 2, 1200, 1200′, or any other processor discussed herein) may involve, in one or more embodiments, a single press with a finger and dragging on the area to make the selection or modification. The new orientation and updates to the view may be calculated upon release of a finger, or a pointer. In one or more embodiments, a region of interest and/or a position of the marker may be set or selected automatically using AI features and/or processing features of the present disclosure.

For one or more embodiments using a touch screen, two simultaneous touch points may be used to make a selection or modification, and may update the view based on calculations upon release.

One or more functions may be controlled with one of the imaging modalities, such as the angiography image view or the intravascular image view (e.g., the OCT image view, the IVUS image view, another intravascular imaging modality image view, etc.), to centralize user attention, maintain focus, and allow the user to see all relevant information in a single moment in time.

In one or more embodiments, one imaging modality may be displayed or multiple imaging modalities may be displayed.

One or more procedures may be used in one or more embodiments to select a region of choice or a region of interest for a view. For example, after a single touch is made on a selected area (e.g., by using a touch screen, by using a mouse or other input device to make a selection, etc.), the semi-circle (or other geometric shape used for the designated area) may automatically adjust to the selected region of choice or interest. Two (2) single touch points may operate to connect/draw the region of choice or interest.

There are many ways to compute intensity, viscosity, resolution (including increasing resolution of one or more images), etc., to use one or more imaging modalities, to construct or reconstruct images or structure(s), to detect tissue and/or characterize tissue, and/or related methods for same, discussed herein, digital as well as analog. In at least one embodiment, a computer, such as the console or computer 1200, 1200′, may be dedicated to control and monitor the imaging (e.g., OCT, single mode OCT, multimodal OCT, multiple imaging modalities, IVUS imaging modality, another intravascular imaging modality discussed herein or known to those skilled in the art, etc.) devices, systems, methods and/or storage mediums described herein.

The electric signals used for imaging may be sent to one or more processors, such as, but not limited to, a computer or processor 2 (see e.g., FIG. 1A), a computer 1200 (see e.g., FIGS. 7A-7C, 9, and 11), a computer 1200′ (see e.g., FIGS. 10 and 11), etc. as discussed further below, via cable(s) or wire(s), such as, but not limited to, the cable(s) or wire(s) 113 (see FIG. 9). Additionally or alternatively, the electric signals, as aforementioned, may be processed in one or more embodiments as discussed above by any other computer or processor or components thereof. The computer or processor 2 as shown in FIG. 1A may be used instead of any other computer or processor discussed herein (e.g., computer or processors 1200, 1200′, etc.), and/or the computer or processor 1200, 1200′ may be used instead of any other computer or processor discussed herein (e.g., computer or processor 2). In other words, the computers or processors discussed herein are interchangeable, and may operate to perform any of the multiple imaging modalities feature(s) and method(s) discussed herein, including using, controlling, and changing a GUI or multiple GUI's and/or performing tissue characterization, tissue detection, and coregistration.

Various components of a computer system 1200 are provided in FIG. 9. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., including but not limited to, being connected to the console, the probe, the imaging apparatus or system, any motor discussed herein, a light source, etc.). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a device or system, such as, but not limited to, an apparatus or system using one or more imaging modalities and related method(s) as discussed herein), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The system 1200 may include one or more additional processors in addition to CPU 1201, and such processors, including the CPU 1201, may be used for tissue or object characterization, diagnosis, evaluation, imaging, construction or reconstruction, and/or coregistration. The system 1200 may further include one or more processors connected via a network connection (e.g., via network 1206). The CPU 1201 and any additional processor being used by the system 1200 may be located in the same telecom network or in different telecom networks (e.g., performing feature(s), function(s), technique(s), method(s), etc. discussed herein may be controlled remotely).

The I/O or communication interface 1205 provides communication interfaces to input and output devices, which may include a light source, a spectrometer, a microphone, a communication cable and a network (either wired or wireless), a keyboard 1210, a mouse (see e.g., the mouse 1211 as shown in FIG. 10), a touch screen or screen 1209, a light pen and so on. The communication interface of the computer 1200 may connect to other components discussed herein via line 113 (as diagrammatically shown in FIG. 9). The Monitor interface or screen 1209 provides communication interfaces thereto.

Any methods and/or data of the present disclosure, such as the methods for performing tissue or object characterization, diagnosis, examination, imaging (including, but not limited to, increasing image resolution, performing imaging using one or more imaging modalities, viewing or changing one or more imaging modalities and related methods (and/or option(s) or feature(s)), etc.), tissue detection, and/or coregistration, for example, as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 1), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, systems, and computer-readable storage mediums related to the processors, such as, but not limited to, the processor of the aforementioned computer 1200, etc., as described above may be achieved utilizing suitable hardware, such as that illustrated in the figures. Functionality of one or more aspects of the present disclosure may be achieved utilizing suitable hardware, such as that illustrated in FIG. 9. Such hardware may be implemented utilizing any of the known technologies, such as standard digital circuitry, any of the known processors that are operable to execute software and/or firmware programs, one or more programmable digital devices or systems, such as programmable read only memories (PROMs), programmable arraylogic devices (PALs), etc. The CPU 1201 (as shown in FIG. 9), the processor or computer 2 (as shown in FIG. 1A) and/or the computer or processor 1200′ (as shown in FIG. 10) may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 2, 1200, 1200′, etc.) may include the aforementioned CPU structure, or may be connected to such CPU structure for communication therewith.

As aforementioned, hardware structure of an alternative embodiment of a computer or console 1200′ is shown in FIG. 10 (see also, FIG. 11). The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid state drive (SSD) 1207. The computer or console 1200′ may include a display 1209. The computer 1200′ may connect with a motor, a console, or any other component of the device(s) or system(s) discussed herein via the operation interface 1214 or the network interface 1212 (e.g., via a cable or fiber, such as the cable or fiber 113 as similarly shown in FIG. 9). A computer, such as the computer 1200′, may include a motor or motion control unit (MCU) in one or more embodiments. The operation interface 1214 is connected with an operation unit such as a mouse device 1211, a keyboard 1210 or a touch panel device. The computer 1200′ may include two or more of each component.

At least one computer program is stored in the SSD 1207, and the CPU 1201 loads the at least one program onto the RAM 1203, and executes the instructions in the at least one program to perform one or more processes described herein, as well as the basic input, output, calculation, memory writing and memory reading processes.

The computer, such as the computer 2, the computer 1200, 1200′, (or other component(s) such as, but not limited to, the PCU, etc.), etc. may communicate with an MCU, an interferometer, a spectrometer, a detector, etc. to perform imaging, and may reconstruct an image from the acquired intensity data. The monitor or display 1209 displays the reconstructed image, and may display other information about the imaging condition or about an object to be imaged. The monitor 1209 also provides a graphical user interface for a user to operate any system discussed herein. An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs any system discussed herein to set or change the imaging condition (e.g., improving resolution of an image or images), and to start or end the imaging. A light or laser source and a spectrometer and/or detector may have interfaces to communicate with the computers 1200, 1200′ to send and receive the status information and the control signals.

As shown in FIG. 11, one or more processors or computers 1200, 1200′ (or any other processor discussed herein) may be part of a system in which the one or more processors or computers 1200, 1200′ (or any other processor discussed herein) communicate with other devices (e.g., a database 1603, a memory 1602 (which may be used with or replaced by any other type of memory discussed herein or known to those skilled in the art), an input device 1600, an output device 1601, etc.). In one or more embodiments, one or more models may have been trained previously and stored in one or more locations, such as, but not limited to, the memory 1602, the database 1603, etc. In one or more embodiments, it is possible that one or more models and/or data discussed herein (e.g., training data, testing data, validation data, imaging data, etc.) may be input or loaded via a device, such as the input device 1600. In one or more embodiments, a user may employ an input device 1600 (which may be a separate computer or processor, a keyboard such as the keyboard 1210, a mouse such as the mouse 1211, a microphone, a screen or display 1209 (e.g., a touch screen or display), or any other input device known to those skilled in the art). In one or more system embodiments, an input device 1600 may not be used (e.g., where user interaction is eliminated by one or more artificial intelligence features discussed herein). In one or more system embodiments, the output device 1601 may receive one or more outputs discussed herein to perform the marker detection, the coregistration, and/or any other process discussed herein. In one or more system embodiments, the database 1603 and/or the memory 1602 may have outputted information (e.g., trained model(s), detected marker information, image data, test data, validation data, training data, coregistration result(s), segmentation model information, object detection/regression model information, combination model information, etc.) stored therein. That said, one or more embodiments may include several types of data stores, memory, storage media, etc. as discussed above, and such storage media, memory, data stores, etc. may be stored locally or remotely.

Additionally, unless otherwise specified, the term “subset” of a corresponding set does not necessarily represent a proper subset and may be equal to the corresponding set.

While one or more embodiments of the present disclosure include various details regarding a neural network model architecture and optimization approach, in one or more embodiments, any other model architecture, machine learning algorithm, or optimization approach may be employed. One or more embodiments may utilize hyper-parameter combination(s). One or more embodiments may employ data capture, selection, annotation as well as model evaluation (e.g., computation of loss and validation metrics) since data may be domain and application specific. In one or more embodiments, the model architecture may be modified and optimized to address a variety of computer visions issues (discussed below).

One or more embodiments of the present disclosure may automatically detect (predict a spatial location of) a radiodense OCT marker in a time series of X-ray images to co-register the X-ray images with the corresponding OCT images (at least one example of a reference point of two different coordinate systems). One or more embodiments may use deep (recurrent) convolutional neural network(s), which may improve marker detection, tissue detection, tissue characterization, and image co-registration significantly. One or more embodiments may employ segmentation and/or object/keypoint detection architectures to solve one or more computer vision issues in other domain areas in one or more applications. One or more embodiments employ several novel materials and methods to solve one or more computer vision or other issues (e.g., radiodense OCT marker detection in time series of X-ray images, for instance; tissue detection; tissue characterization; etc.).

One or more embodiments employ data capture and selection. In one or more embodiments, the data is what makes such an application unique and distinguishes this application from other applications. For example, images may include a radiodense marker that is specifically used in one or more procedures (e.g., added to the OCT capsule, used in catheters/probes with a similar marker to that of an OCT marker, used in catheters/probes with a similar or same marker even in a case where the catheters/probes use an imaging modality different from OCT, etc.) to facilitate computational detection of a marker and/or tissue detection, characterization, validation, etc. in one or more images (e.g., X-ray images). One or more embodiments may couple a software device or features (model) to hardware (e.g., an OCT probe, a probe/catheter using an imaging modality different from OCT while using a marker that is the same as or similar to the marker of an OCT probe/catheter, etc.). One or more embodiments may utilize animal data in addition to patient data. Training deep learning may use a large amount of data, which may be difficult to obtain from clinical studies. Inclusion of image data from pre-clinical studies in animals into a training set may improve model performance. Training and evaluation of a model may be highly data dependent (e.g., a way in which frames are selected (e.g., pullback only), split into training/validation/test sets, and grouped into batches as well as the order in which the frames, sets, and/or batches are presented to the model, any other data discussed herein, etc.). In one or more embodiments, such parameters may be more important or significant than some of the model hyper-parameters (e.g., batch size, number of convolution layers, any other hyper-parameter discussed herein, etc.). One or more embodiments may use a collection or collections of user annotations after introduction of a device/apparatus, system, and/or method(s) into a market, and may use post market surveillance, retraining of a model or models with new data collected (e.g., in clinical use), and/or a continuously adaptive algorithm/method(s).

One or more embodiments may employ data annotation. For example, one or more embodiments may label pixel(s) representing a marker or a tissue detection, characterization, and/or validation as well as pixels representing a blood vessel(s) at different phase(s) of a procedure/method (e.g., different levels of contrast due to intravascular contrast agent) of frame(s) acquired during pullback.

One or more embodiments may employ incorporation of prior knowledge. For example, in one or more embodiments, a marker location may be known inside a vessel and/or inside a catheter or probe; a tissue location may be known inside a vessel or other type of target, object, or specimen; etc. As such, simultaneous localization of the vessel and marker may be used to improve marker detection and/or tissue detection, characterization, and/or validation. For example, in a case where it is confirmed that the marker of the probe or catheter, or the catheter or probe, is by or near a target area for tissue detection and characterization, the integrity of the tissue identification/detection and/or characterization for that target area is improved or maximized (as compared to a false positive where a tissue may be detected in an area where the probe or catheter (or marker thereof) is not located). In one or more embodiments, a marker may move during a pullback inside a vessel, and such prior knowledge may be incorporated into the machine learning algorithm or the loss function.

One or more embodiments employ loss (cost) and evaluation function(s)/metric(s). For example, use of temporal information for model training and evaluation may be used in one or more embodiments. One or more embodiments may evaluate a distance between prediction and ground truth per frame as well as consider a trajectory of predictions across multiple frames of a time series.

### Application of Machine Learning

Application of machine learning may be used in one or more embodiment(s), as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, at least one embodiment of an overall process of machine learning is shown below:
i. Create a dataset that contains both images and corresponding ground truth labels;
ii. Split the dataset into a training set and a testing set;
iii. Select a model architecture and other hyper-parameters;
iv. Train the model with the training set;
v. Evaluate the trained model with the validation set; and
vi. Repeat iv and v with new dataset(s).

Based on the testing results, steps i and iii may be revisited in one or more embodiments.

One or more models may be used in one or more embodiment(s) to detect and/or characterize a tissue or tissues, such as, but not limited to, the one or more models as discussed in PCT/US2020/051615, filed on Sep. 18, 2020 and published as WO 2021/055837 A9 on Mar. 25, 2021, and as discussed in U.S. patent application Ser. No. 17/761,561, filed on Mar. 17, 2022, the applications and publications of which are incorporated by reference herein in their entireties. For example, one or more embodiments may use a segmentation model, a regression model, a combination thereof, etc.

For regression model(s), the input may be the entire image frame or frames, and the output may be the centroid coordinates of radiopaque markers (target marker and stationary marker, if necessary/desired) and/or coordinates of a portion of a catheter or probe to be used in determining the tissue detection and/or characterization. Additionally or alternatively, in one or more embodiments, input may comprise or include an entire image frame or frames (e.g., the aforementioned constructed CVI image or frame), and the output may be data regarding high textured areas formed due to the presence of sharp edges in an A-line or A-lines representing calcium in intravascular images as well as dark homogeneous areas representing lipids in the input image frame or frames (e.g., in the CVI image or frame). As shown diagrammatically in FIGS. 12-14, an example of an input image on the left side of FIGS. 12-14 and a corresponding output image on the right side of FIGS. 12-14 are illustrated for regression model(s). At least one architecture of a regression model is shown in FIG. 12. In at least the embodiment of FIG. 12, the regression model may use a combination of one or more convolution layers 900, one or more max-pooling layers 901, and one or more fully connected dense layers 902. While not limited to the Kernel size, Width/Number of filters (output size), and Stride sizes shown for each layer (e.g., in the left convolution layer of FIG. 12, the Kernel size is “3×3”, the Width/# of filters (output size) is “64”, and the Stride size is “2”). In one or more embodiments, another hyper-parameter search with a fixed optimizer and with a different width may be performed, and at least one embodiment example of a model architecture for a convolutional neural network for this scenario is shown in FIG. 13. One or more embodiments may use one or more features for a regression model as discussed in “Deep Residual Learning for Image Recognition” to Kaiming He, et al., Microsoft Research, Dec. 10, 2015 (https://arxiv.org/pdf/1512.03385.pdf), which is incorporated by reference herein in its entirety. FIG. 14 shows at least a further embodiment example of a created architecture of or for a regression model(s).

Since the output from a segmentation model, in one or more embodiments, is a “probability” of each pixel that may be categorized as a tissue characterization or tissue identification/determination, post-processing after prediction via the trained segmentation model may be developed to better define, determine, or locate the final coordinate of tissue location (or a marker location where the marker is a part of the catheter) and/or determine the type and/or characteristics of the tissue or tissues. One or more embodiments of a semantic segmentation model may be performed using the One-Hundred Layers Tiramisu method discussed in “The One Hundred Layers Tiramisu: Fully Convolutional DenseNets for Semantic Segmentation” to Simon Jégou, et al., Montreal Institute for Learning Algorithms, published Oct. 31, 2017 (https://arxiv.org/pdf/1611.09326.pdf), which is incorporated by reference herein in its entirety. A segmentation model may be used in one or more embodiment, for example, as shown in FIG. 15. At least one embodiment may utilize an input 600 as shown to obtain an output 605 of at least one embodiment of a segmentation model method. For example, by applying the One-Hundred Layers Tiramisu method(s), one or more features, such as, but not limited to, convolution 601, concatenation 603, transition up 605, transition down 604, dense block 602, etc., may be employed by slicing the training data set. While not limited to only or by only these embodiment examples, in one or more embodiments, a slicing size may be one or more of the following: 100×100, 224×224, 512×512, and, in one or more of the experiments performed, a slicing size of 224×224 performed the best. A batch size (of images in a batch) may be one or more of the following: 2, 4, 8, 16, and, from the one or more experiments performed, a bigger batch size typically performs better (e.g., with greater accuracy). In one or more embodiments, 16 images/batch may be used. The optimization of all of these hyper-parameters depends on the size of the available data set as well as the available computer/computing resources; thus, once more data is available, different hyper-parameter values may be chosen. Additionally, in one or more embodiments, steps/epoch may be 100, and the epochs may be greater than (>) 1000. In one or more embodiments, a convolutional autoencoder (CAE) may be used.

In one or more embodiments, hyper-parameters may include, but are not limited to, one or more of the following: Depth (i.e., # of layers), Width (i.e., # of filters), Batch size (i.e., # of training images/step): May be >4 in one or more embodiments, Learning rate (i.e., a hyper-parameter that controls how fast the weights of a neural network (the coefficients of regression model) are adjusted with respect the loss gradient), Dropout (i.e., % of neurons (filters) that are dropped at each layer), and/or Optimizer: for example, Adam optimizer or Stochastic gradient descent (SGD) optimizer. In one or more embodiments, other hyper-parameters may be fixed or constant values, such as, but not limited to, for example, one or more of the following: Input size (e.g., 1024 pixel×1024 pixel, 512 pixel×512 pixel, another preset or predetermined number or value set, etc.), Epochs: 100, 200, 300, 400, 500, another preset or predetermined number, etc. (for additional training, iteration may be set as 3000 or higher), and/or Number of models trained with different hyper-parameter configurations (e.g., 10, 20, another preset or predetermined number, etc.).

One or more features discussed herein may be determined using a convolutional auto-encoder, Gaussian filters, Haralick features, and/or thickness or shape of the sample or object (e.g., the tissue or tissues, a specimen, a patient, a target in the patient, etc.).

One or more embodiments of the present disclosure may use machine learning to determine marker or tissue location; to determine, detect, or evaluate tissue type(s) and/or characteristic(s); to perform coregistration; and/or to perform any other feature discussed herein. Machine learning (ML) is a field of computer science that gives processors the ability to learn, via artificial intelligence. Machine learning may involve one or more algorithms that allow processors or computers to learn from examples and to make predictions for new unseen data points. In one or more embodiments, such one or more algorithms may be stored as software or one or more programs in at least one memory or storage medium, and the software or one or more programs allow a processor or computer to carry out operation(s) of the processes described in the present disclosure.

Similarly, the present disclosure and/or one or more components of devices, systems, and storage mediums, and/or methods, thereof also may be used in conjunction with optical coherence tomography probes. Such probes include, but are not limited to, the OCT imaging systems disclosed in U.S. Pat. Nos. 6,763,261; 7,366,376; 7,843,572; 7,872,759; 8,289,522; 8,676,013; 8,928,889; 9,087,368; 9,557,154; 10,912,462; 9,795,301; and U.S. Pat. No. 9,332,942 to Tearney et al., and U.S. Pat. Pub. Nos. 2014/0276011 and 2017/0135584; and WO 2016/015052 to Tearney et al., and arrangements and methods of facilitating photoluminescence imaging, such as those disclosed in U.S. Pat. No. 7,889,348 to Tearney et al., as well as the disclosures directed to multimodality imaging disclosed in U.S. Pat. No. 9,332,942, and U.S. Patent Publication Nos. 2010/0092389, 2011/0292400, 2012/0101374, 2016/0228097, 2018/0045501 and 2018/0003481, and WO 2016/144878, each of which patents and patent publications are incorporated by reference herein in their entireties. As aforementioned, any feature or aspect of the present disclosure may be used with OCT imaging systems, apparatuses, methods, storage mediums or other aspects or features as discussed in U.S. patent application Ser. No. 16/414,222, filed on May 16, 2019 and published on Dec. 12, 2019 as U.S. Pat. Pub. No. 2019/0374109, the entire disclosure of which is incorporated by reference herein in its entirety.

The present disclosure and/or one or more components of devices, systems, and storage mediums, and/or methods, thereof also may be used in conjunction with OCT imaging systems and/or catheters and catheter systems, such as, but not limited to, those disclosed in U.S. Pat. Nos. 9,869,828; 10,323,926; 10,558,001; 10,601,173; 10,606,064; 10,743,749; 10,884,199; 10,895,692; and 11,175,126 as well as U.S. Patent Publication Nos. 2019/0254506; 2020/0390323; 2021/0121132; 2021/0174125; 2022/0040454; 2022/0044428, and WO2021/055837, each of which patents and patent publications are incorporated by reference herein in their entireties.

Further, the present disclosure and/or one or more components of devices, systems, and storage mediums, and/or methods, thereof also may be used in conjunction with continuum robotic systems and catheters, such as, but not limited to, those described in U.S. Patent Publication Nos. 2019/0105468; 2021/0369085; 2020/0375682; 2021/0121162; 2021/0121051; and 2022-0040450, each of which patents and/or patent publications are incorporated by reference herein in their entireties.

Although the disclosure herein has been described with reference to particular embodiments, it is to be understood that these embodiments are merely illustrative of the principles and applications of the present disclosure (and are not limited thereto). It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications and equivalent structures and functions.

1. An apparatus for detecting and/or characterizing one or more tissues in one or more images, the apparatus comprising:
one or more processors that operate to:
(i) perform a pullback of a catheter or probe and/or obtain one or more images or frames from the pullback of the catheter or probe;
(ii) create or construct a Carpet View Image (CVI) based on the one or more images or frames from the pullback or otherwise receive or obtain the CVI;
(iii) detect or identify tissue type(s) of one or more tissues shown in the CVI, and/or determine one or more characteristics of the one or more tissues, including whether the one or more tissues is a calcium, a lipid, or another type of tissue;
(iv) update the CVI by overlaying information on the CVI to indicate the detected or identified tissue type(s) and/or the determined one or more characteristics of the one or more tissues; and
(v) display the updated CVI, or the updated CVI with one or more images or frames from the pullback on a display, or store the updated CVI in a memory.

2. The apparatus of claim 1, wherein the one or more processors further operate to one or more of the following:
(i) detect one or more tissue types automatically in the pullback of the catheter or the probe for one or more intravascular or Optical Coherence Tomography (OCT) images, where the one or more tissue types include the calcium type, the lipid(s) type, a fibrous tissue type, a mixed tissue type, or the another tissue type;
(ii) reduce computational time to characterize the pullback by processing one image only, where the CVI is the one image;
(iii) not require any segmentation prior to tissue characterization;
(iv) perform a more detailed tissue detection or characterization based on a spatial connection or connections of tissue in adjacent frames of the pullback, and/or with each pixel characterization that is based on values of its neighborhood or neighboring pixels being taken into consideration, and/or based on the spatial connection(s) and/or neighboring or neighborhood pixel(s) consideration being characterized by artificial intelligence, Machine Learning (ML), and/or deep learning networks, structure, or algorithm(s) useable by the one or more processors;
(v) use A-line based approaches so that a length of a tissue arc is calculated by the one or more processors; and/or
(vi) use pixel based approaches so that a tissue area is quantified by the one or more processors.

3. The apparatus of claim 1, wherein the one or more processors further operate to perform one or more of the following:
display the CVI for further processing and/or display the CVI with one or more intravascular or Optical Coherence Tomography (OCT) images;
co-register high texture carpet view areas of the CVI with one or more intravascular or Optical Coherence Tomography (OCT) images;
overlay a line on one or more intravascular or Optical Coherence Tomography (OCT) images corresponding to a border indicating the presence of a first type of tissue or the calcium where the line is either a solid line or a dashed or dotted line, and/or overlay a different line on the one or more intravascular or OCT images corresponding to a border indicating the presence of a second type of tissue or the lipid(s) where the different line is the other of the solid line or the dashed or dotted line; and/or
display two copies of the CVI, where a first copy of the CVI includes lines overlaid on a first copy of the CVI where each of the lines indicate a border in one or more intravascular or Optical Coherence Tomography (OCT) images and where a second copy of the CVI includes first annotated area(s) corresponding to a first tissue or calcified area(s) and second annotated area(s) corresponding to a second tissue or lipid area(s).

4. The apparatus of claim 3, wherein the one or more processors further operate to:
in a case where the high texture carpet view areas are detected by the one or more processors, form the high texture carpet view areas due to a presence of sharp edges in the A-line frames or images which represent calcium in the one or more intravascular or OCT images; and
in a case where dark homogenous areas are detected by the one or more processors, form or associate corresponding dark homogenous areas to represent lipid(s).

5. The apparatus of claim 4, wherein the one or more processors further operate to use AI network(s) and Machine Learning (ML) or other AI-based features to train models to automatically detected calcium and/or lipids based on the use of the high texture carpet view areas representing calcium and based on the dark homogenous areas representing lipid(s).

6. The apparatus of claim 5, wherein the one or more processors further operate to use the trained models and/or trained AI network(s) on the CVI only to determine or identify and/or characterize the tissue or tissue characteristics.

7. The apparatus of claim 1, wherein the one or more processors further operate to:
construct a patch of length L around each pixel of the CVI;
extract a set of intensity and texture features from the patch or pixel and classify the pixel to the lipid tissue type, the calcium tissue type, or the another tissue type using artificial intelligence, where the artificial intelligence is one of: Machine Learning (ML), random forests, support vector machines (SVM), and/or another AI-based method, network, or feature; or auto-extract and auto-define a set of intensity and texture features from the patch or pixel using a neural network, convolutional neural network, or other AI-based method or feature and classify the pixel to the lipid tissue type, the calcium tissue type, or the another tissue type; and
point, mark, or otherwise indicate, in one or more intravascular or Optical Coherence Tomography (OCT) images, where the calcium and/or lipid starts and ends by using a column part of each corresponding area detected by the one or more processors.

8. The apparatus of claim 7, wherein the one or more processors further operate to one or more of the following:
(i) set a pixel value of 1;
(ii) perform construction of the patch based on the CVI;
(iii) perform AI processing by using an AI network or other AI structure to obtain or generate pre-trained classifier(s) or patch feature extraction(s) and to obtain or generate a pre-trained Machine Learning (ML) classifier(s);
(iv) identify or characterize whether the pixel being evaluated for the patch construction is a calcium pixel, a lipid pixel, or another type of pixel;
(v) perform calcium and lipid pixel translation to a cross sectional intravascular or Optical Coherence Tomography (OCT) image or frame and/or to the one or more intravascular or OCT images; and/or
(vi) determine whether the pixel value is less than a value for the number of A-lines x, or by, the number of pullback frames, and, in a case where the pixel value is less than the value for the number of A-lines x, or by, the number of pullback frames, then add 1 to the pixel value and repeat limitations (ii) through (vi) for the next pixel being evaluated, or in a case where the pixel value is greater than or equal to the value for the number of A-lines x, or by, the number of pullback frames, then complete the tissue characterization.

9. The apparatus of claim 8, wherein the one or more processors further operate to display results of the tissue characterization completion on the display, store the results in the memory, or use the results to train one or more models or AI-networks to auto-detect or auto-characterize the tissue.

10. The apparatus of claim 9, wherein the trained model is one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel and/or including tissue characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s).

11. The apparatus of claim 8, wherein the one or more processors further operate to:
indicate a calcium pixel or patch using a solid line and/or indicate a lipid pixel or patch using a dotted or dashed line overlaid on the one or more intravascular or OCT images and/or on the CVI; and/or
perform a more detailed tissue detection by taking into consideration a spatial connection or connections of tissue in adjacent frames of the pullback.

12. The apparatus of claim 1, wherein:
the CVI has dimensions equal to a number of A-lines (A) of each A-line frame times, or by, a number of pullback cross sectional frames or images or by a number of total frames (N), and setting a counter i to a value of 1; and
the one or more processors further operate to:
(i) determine whether i is less than or equal to the number of pullback frames, N; and
(ii) in a case where i is less than or equal to N and until i is more than N, repeat the performance of the following:
acquire an A-line frame corresponding to the value for i;
perform or apply thresholding or automatic thresholding for the acquired A-line frame or image;
summarize each line of the thresholded image or frame, and/or compress the thresholded image or frame in one dimension (1D) by summing columns of the thresholded image or frame so that each line of the thresholded image or frame is in 1D; and
add the 1D line to the ith line of the created or constructed CVI, and add 1 to the value for i; and/or
(iii) in a case where i is more than N such that all of the pullback frames, N, have been processed, show, reveal, display on a display, and/or store the created or constructed CVI in the memory.

13. The apparatus of claim 12, wherein the constructed or created CVI is saved in the memory and/or is sent to the one or more processors or an artificial intelligence (AI) network for use in AI evaluations or determinations.

14. The apparatus of claim 13, wherein the one or more processors further operate to use one or more neural networks or convolutional neural networks to one or more of: load a trained model of CVI images including calcium and or lipid area(s), create or construct the CVI, evaluate whether the counter i is less than or equal to, or greater than, the number of pullback frames N, detect or identify the tissue type(s) in the CVI, apply the thresholding or automatic thresholding, perform the summarizing or compressing of the thresholded image in 1D by summing the columns of the image, perform the addition of the 1D line to the ith line of the CVI, determine whether the detected or identified tissue type(s) is/are accurate or correct, determine the one or more of the characteristics of the tissue(s), identify or detect the one or more tissues, overlay data on the CVI to show the location(s) of the intravascular image(s) and/or to show the areas for the tissue type(s), display the results for the tissue identification/detection or characterization on a display, and/or acquire or receive the image data during the pullback operation of the catheter or the probe.

15. The apparatus of claim 13, wherein the one or more processors further operate to use one or more neural networks or convolutional neural networks to one or more of: incorporate image processing and machine learning (ML) or deep learning to automatically identify and locate calcium and lipid(s); create or construct the CVI for the whole pullback and apply ML only to the CVI; create or construct the CVI or another image having dimensions equal to the number of A-lines (A) of each A-line frame, by the number of total frames (N); acquire the first A-line frame and continue to acquire the A-lines of the frame, threshold the image, summarize each A-line of the thresholded image to generate a one-dimensional (1D) signal or line having size A, add or copy the 1D signal or line in or to a first column of the A×N image, and repeat the acquire, threshold, summarize, and add or copy features for all of the pullback frames so that a next 1 D signal or line is added or copied in or to the corresponding next column of the A×N image until all subsequent 1D signals or lines are added or copied in or to the corresponding subsequent, respective columns of the A×N image; and reveal, show, or display the CVI or the A×N image, and/or store the created or constructed CVI in the memory, after the last 1D signal or line is copied or added in or to the last column of the A×N image.

16. The apparatus of claim 1, further comprising one or more of the following:
a light source that operates to produce a light;
an interference optical system that operates to: (i) receive and divide the light from the light source into a first light with which an object or sample is to be irradiated and a second reference light, (ii) send the second reference light for reflection off of a reference mirror of the interference optical system, and (iii) generate interference light by causing reflected or scattered light of the first light with which the object or sample has been irradiated and the reflected second reference light to combine or recombine, and to interfere, with each other, the interference light generating one or more interference patterns; and/or
one or more detectors that operate to continuously acquire the interference light and/or the one or more interference patterns such that the one or more lumen edges, the one or more stents, and/or the one or more artifacts are detected in the images, and the one or more stents and/or the one or more artifacts are removed from the one or more images.

17. A method for detecting and/or characterizing one or more tissues in one or more images, the method comprising:
(i) performing a pullback of a catheter or probe and/or obtaining one or more images or frames from the pullback of the catheter or probe;
(ii) creating or constructing a Carpet View Image (CVI) based on the one or more images or frames from the pullback or otherwise receive or obtain the CVI;
(iii) detecting or identifying tissue type(s) of one or more tissues shown in the CVI, and/or determining one or more characteristics of the one or more tissues, including whether the one or more tissues is a calcium, a lipid, or another type of tissue;
(iv) updating the CVI by overlaying information on the CVI to indicate the detected or identified tissue type(s) and/or the determined one or more characteristics of the one or more tissues; and
(v) displaying the updated CVI, or the updated CVI with one or more images or frames from the pullback on a display, and/or storing the updated CVI in a memory.

18. The method of claim 17, further comprising one or more of the following:
(i) detecting one or more tissue types automatically in the pullback of the catheter or the probe for one or more intravascular or Optical Coherence Tomography (OCT) images, where the one or more tissue types include the calcium type, the lipid(s) type, a fibrous tissue type, a mixed tissue type, or the another tissue type;
(ii) reducing computational time to characterize the pullback by processing one image only, where the CVI is the one image;
(iii) not requiring any segmentation prior to tissue characterization;
(iv) performing a more detailed tissue detection or characterization based on a spatial connection or connections of tissue in adjacent frames of the pullback, and/or with each pixel characterization that is based on values of its neighborhood or neighboring pixels being taken into consideration, and/or based on the spatial connection(s) and/or neighboring or neighborhood pixel(s) consideration being characterized by artificial intelligence, Machine Learning (ML), and/or deep learning networks, structure(s), or method(s);
(v) using A-line based approaches so that a length of a tissue arc is calculated; and/or
(vi) using pixel based approaches so that a tissue area is quantified.

19. The method of claim 17, further comprising one or more of the following:
displaying the CVI for further processing and/or displaying the CVI with one or more intravascular or Optical Coherence Tomography (OCT) images;
co-registering high texture carpet view areas of the CVI with one or more intravascular or Optical Coherence Tomography (OCT) images;
overlaying a line on one or more intravascular or Optical Coherence Tomography (OCT) images corresponding to a border indicating the presence of a first type of tissue or the calcium where the line is either a solid line or a dashed or dotted line, and/or overlaying a different line on the one or more intravascular or OCT images corresponding to a border indicating the presence of a second type of tissue or the lipid(s) where the different line is the other of the solid line or the dashed or dotted line; and/or
displaying two copies of the CVI, where a first copy of the CVI includes lines overlaid on a first copy of the CVI where each of the lines indicate a border in one or more intravascular or Optical Coherence Tomography (OCT) images and where a second copy of the CVI includes first annotated area(s) corresponding to a first tissue or calcified area(s) and second annotated area(s) corresponding to a second tissue or lipid area(s).

20. The method of claim 19, further comprising:
in a case where the high texture carpet view areas are detected, forming the high texture carpet view areas due to a presence of sharp edges in the A-line frames or images which represent calcium in the one or more intravascular or OCT images; and
in a case where dark homogenous areas are detected, forming or associating corresponding dark homogenous areas to represent lipid(s).

21. The method of claim 20, further comprising: using AI network(s) and Machine Learning (ML) or other AI-based features to train models to automatically detected calcium and/or lipids based on the use of the high texture carpet view areas representing calcium and based on the dark homogenous areas representing lipid(s).

22. The method of claim 21, further comprising: using the trained models and/or trained AI network(s) on the CVI only to determine or identify and/or characterize the tissue or tissue characteristics.

23. The method of claim 17, further comprising:
constructing a patch of length L around each pixel of the CVI;
extracting a set of intensity and texture features from the patch or pixel and classifying the pixel to the lipid tissue type, the calcium tissue type, or the another tissue type using artificial intelligence, where the artificial intelligence is one of: Machine Learning (ML), random forests, support vector machines (SVM), and/or another AI-based method, network, or feature; or auto-extracting and auto-defining a set of intensity and texture features from the patch or pixel using a neural network, convolutional neural network, or other AI-based method or feature and classifying the pixel to the lipid tissue type, the calcium tissue type, or the another tissue type; and
pointing, marking, or otherwise indicating, in one or more intravascular or Optical Coherence Tomography (OCT) images, where the calcium and/or lipid starts and ends by using a column part of each corresponding detected area.

24. The method of claim 23, further comprising one or more of the following:
(i) setting a pixel value of 1;
(ii) performing construction of the patch based on the CVI;
(iii) performing AI processing by using an AI network or other AI structure to obtain or generate pre-trained classifier(s) or patch feature extraction(s) and to obtain or generate a pre-trained Machine Learning (ML) classifier(s);
(iv) identifying or characterizing whether the pixel being evaluated for the patch construction is a calcium pixel, a lipid pixel, or another type of pixel;
(v) performing calcium and lipid pixel translation to a cross sectional intravascular or Optical Coherence Tomography (OCT) image or frame and/or to the one or more intravascular or OCT images; and/or
(vi) determining whether the pixel value is less than a value for the number of A-lines x, or by, the number of pullback frames, and, in a case where the pixel value is less than the value for the number of A-lines x, or by, the number of pullback frames, then adding 1 to the pixel value and repeating limitations (ii) through (vi) for the next pixel being evaluated, or in a case where the pixel value is greater than or equal to the value for the number of A-lines x, or by, the number of pullback frames, then completing or ending the tissue characterization.

25. The method of claim 24, further comprising displaying results of the tissue characterization completion on the display, storing the results in the memory, or using the results to train one or more models or AI-networks to auto-detect or auto-characterize the tissue.

26. The method of claim 25, wherein the trained model is one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel and/or including tissue characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, and/or a model using repeated object detection or regression model technique(s).

27. The method of claim 24, further comprising:
indicating a calcium pixel or patch using a solid line and/or indicating a lipid pixel or patch using a dotted or dashed line overlaid on the one or more intravascular or OCT images and/or on the CVI; and/or
performing a more detailed tissue detection by taking into consideration a spatial connection or connections of tissue in adjacent frames of the pullback.

28. The method of claim 17, wherein:
the CVI has dimensions equal to a number of A-lines (A) of each A-line frame times, or by, a number of pullback cross sectional frames or images or by a number of total frames (N), and setting a counter i to a value of 1; and
the method further comprises:
(i) determining whether i is less than or equal to the number of pullback frames, N; and
(ii) in a case where i is less than or equal to N and until i is more than N, repeating the performance of the following:
acquiring an A-line frame corresponding to the value for i;
performing or applying thresholding or automatic thresholding for the acquired A-line frame or image;
summarizing each line of the thresholded image or frame, and/or compressing the thresholded image or frame in one dimension (1D) by summing columns of the thresholded image or frame so that each line of the thresholded image or frame is in 1D; and
adding the 1D line to the ith line of the created or constructed CVI, and adding 1 to the value for i; and/or
(iii) in a case where i is more than N such that all of the pullback frames, N, have been processed, showing, revealing, displaying on a display, and/or storing the created or constructed CVI in the memory.

29. The method of claim 28, wherein the constructed or created CVI is saved in the memory and/or is sent to one or more processors or an artificial intelligence (AI) network for use in AI evaluations or determinations.

30. The method of claim 29, further comprising using one or more neural networks or convolutional neural networks to one or more of: load a trained model of CVI images including calcium and or lipid area(s), create or construct the CVI, evaluate whether the counter i is less than or equal to, or greater than, the number of pullback frames N, detect or identify the tissue type(s) in the CVI, apply the thresholding or automatic thresholding, perform the summarizing or compressing of the thresholded image in 1D by summing the columns of the image, perform the addition of the 1D line to the ith line of the CVI, determine whether the detected or identified tissue type(s) is/are accurate or correct, determine the one or more of the characteristics of the tissue(s), identify or detect the one or more tissues, overlay data on the CVI to show the location(s) of the intravascular image(s) and/or to show the areas for the tissue type(s), display the results for the tissue identification/detection or characterization on a display, and/or acquire or receive the image data during the pullback operation of the catheter or the probe.

31. The method of claim 29, further comprising: using one or more neural networks or convolutional neural networks to one or more of: incorporate image processing and machine learning (ML) or deep learning to automatically identify and locate calcium and lipid(s); create or construct the CVI for the whole pullback and apply ML only to the CVI; create or construct the CVI or another image having dimensions equal to the number of A-lines (A) of each A-line frame, by the number of total frames (N); acquire the first A-line frame and continue to acquire the A-lines of the frame, threshold the image, summarize each A-line of the thresholded image to generate a one-dimensional (1D) signal or line having size A, add or copy the 1D signal or line in or to a first column of the A×N image, and repeat the acquire, threshold, summarize, and add or copy features for all of the pullback frames so that a next 1 D signal or line is added or copied in or to the corresponding next column of the A×N image until all subsequent 1D signals or lines are added or copied in or to the corresponding subsequent, respective columns of the A×N image; and reveal, show, or display the CVI or the A×N image, and/or store the created or constructed CVI in the memory, after the last 1D signal or line is copied or added in or to the last column of the A×N image.

32. A computer-readable storage medium storing at least one program that operates to cause one or more processors to execute a method for detecting and/or characterizing one or more tissues in one or more images, the method comprising:
(i) performing a pullback of a catheter or probe and/or obtaining one or more images or frames from the pullback of the catheter or probe;
(ii) creating or constructing a Carpet View Image (CVI) based on the one or more images or frames from the pullback or otherwise receive or obtain the CVI;
(iii) detecting or identifying tissue type(s) of one or more tissues shown in the CVI, and/or determining one or more characteristics of the one or more tissues, including whether the one or more tissues is a calcium, a lipid, or another type of tissue;
(iv) updating the CVI by overlaying information on the CVI to indicate the detected or identified tissue type(s) and/or the determined one or more characteristics of the one or more tissues; and
(v) displaying the updated CVI, or the updated CVI with one or more images or frames from the pullback on a display, and/or storing the updated CVI in a memory.
